(19) United States
(12) Patent Application Publication
(10) Pub. No.: US 2008/0176298 A1
(43) Pub. Date: Jul. 24, 2008

(54) OIL RECOVERY FROM DRY CORN MILLING ETHANOL PRODUCTION PROCESSES

(76) Inventors: Sarabjit S. Randhava, Evanston, IL (US); Richard L. Kao, Northbrook, IL (US); Steven G. Calderone, Oak Park, IL (US); Ajaib S. Randhava, Streamwood, IL (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/939,191

(22) Filed: Nov. 13, 2007

Related U.S. Application Data
(60) Provisional application No. 60/858,960, filed on Nov. 15, 2006.

(51) Int. Cl. C12P 7/64; C12M 1/00

(52) U.S. Cl. 435/134; 435/291.1

(57) ABSTRACT

A corn oil extraction process is disclosed. The process includes the recovery of corn oil and other co-products, including but not limited to steam, electric power and chemicals, from an ethanol production process and in particular, a process that involves dry corn milling methods. The process involves extraction of oil from milled corn and residues from the fermentation step, including thick stillage, distillers wet grain, distillers dry grain and distillers dry grains with solubles, by the application of an alkyl acetate, phase separation and recovery of the separated matter. A process of drying wet co-product using ethanol and carbon dioxide from the production facility is also disclosed.

### CROSS-REFERENCE TO RELATED APPLICATIONS

This application claims priority to U.S. Provisional Patent Application Ser. No. 60/858,960 entitled “Oil Recovery from Dry Corn Milling Ethanol Production Processes”, filed Nov. 15, 2006, the entire contents of which is hereby incorporated by reference in its entirety.

### SCOPE OF THE INVENTION

The present invention relates to corn technologies and more specifically to the extraction of oil and other co-products from corn associated with the ethanol production process.

### BACKGROUND

As is known, in the production of ethanol from dry milled corn, various by-products or co-products are produced.

In the production of ethanol, after fermentation, it is known to continuously pump beer from fermentors into a still that serves to separate the alcohol from the co-products. Typically, in the process, from the distillation column, which often includes 6-16 wt % or greater total solids (typically, 14 wt % of solids are in liquid, either suspended or dissolved), the non-volatile suspended and dissolved solids in the feed are washed down through a lower stripping section and a stream, the thick stillage (TS) containing less than 0.02 wt % ethanol is removed from the bottom of the tower or distillation column. The temperature of this stream exiting the distillation column is quite high. For instance, even after some heat recovery, the exit temperature of the stream pumped out of the distillation column ranges from 95-99° C. As indicated, this TS exit stream typically contains approximately 14 wt % solids. Two-thirds of these solids generally exist as a suspension, the remainder being dissolved in liquid. The TS stream is typically centrifuged and separated into two independent streams, one containing the suspended solids (typically around 35 wt %) and the other stream, the thin stillage, containing water and dissolved solids. Each stream is progressively dried to yield the desired products.

As is known, the TS may be further processed. For example, the suspended solids stream containing approximately 35 wt % solids called distillers wet grains (DWG), typically has a shelf life of approximately 3 to 5 days and can only be sold to farm operations in the immediate vicinity of an ethanol plant. The stream may be dried to produce distillers modified wet grains (DMWG), containing roughly 50 wt % solids, which typically has a shelf life of about 30 days and can only be sold in regional markets within the region of the ethanol plant. Alternatively, the stream can be further dried to produce distillers dry grains (DDG), having about 90 wt % solids. Typically, at this stage the stream has been dried to roughly 10 wt % or less water and typically has a shelf life of 2-5 years. This product is sold and shipped throughout the world.

The thin stillage, which includes dissolved solubles in water, may also be further processed. For instance, the thin stillage stream may be dried to produce condensed distillers solubles (CDS), which includes about 35 wt % solids and has a short shelf life. This product is typically blended with DWG for sale. The thin stillage can be further dried to form modified distillers solubles (MDS), containing roughly 50 wt % solids and typically has a 6 month shelf life when stored in a CO₂ blanket bladder. This product is typically blended with DMWG for sale. Thin stillage may be further dried to form distillers dried solubles (DDS), containing about 90 wt % solids, which has a one year shelf life. DDS can be sold independently, or can be combined with DDG to form distillers dried grains with solubles (DDGS) for sale.

Each of the foregoing products are sold primarily as feed. Thus, many of the co-products typically produced from the ethanol production process and sold have a limited shelf life and are of limited value and market.

Accordingly, what is needed in the art is a process of efficiently and effectively obtaining additional co-products from the ethanol production process both upstream and downstream of fermentation, to improve value gained from corn and reduce waste.

### SUMMARY OF THE INVENTION

A process for extracting corn oil from corn in an ethanol production process is disclosed. The process comprises obtaining a corn-based product from the ethanol production process, application of an alkyl acetate solvent to the corn-based product to extract oil so as to produce an extraction solution of at least corn-based product solids, oil, solvent and water, separating the extraction solution into a first phase containing solvent and oil and a second phase containing at least one of water and solids, separating the first phase from the second phase and removing the solvent from the oil. Application of the alkyl acetate solvent may occur prior to fermentation in the ethanol production process, or post fermentation in the ethanol production process in which it is applied to at least one byproduct of the fermentation process. These byproducts include, but are not limited to, TS, DWG and DDGS.

### BRIEF DESCRIPTION OF THE DRAWINGS

FIG. 1 illustrates a traditional co-product flow chart of an ethanol production process, post fermentation.

FIG. 2 illustrates a process flow chart showing the alternate extraction locations utilized in a preferred embodiment of the present invention.

FIG. 3 illustrates a process flow chart according to one embodiment of the present invention in which extraction of oil is from the milled corn.

FIG. 4 illustrates an alternative process flow chart for a TS stream according to an embodiment of the present invention.

FIG. 5 illustrates a process flow chart according to an alternative embodiment of the present invention in which extraction of oil is from the TS.

FIG. 6 illustrates a process flow chart of an ethanol-based filter cake drying process according to one embodiment of the invention.

FIG. 7 illustrates a process flow chart according to an alternative embodiment of the present invention in which extraction of oil is from DWG.

FIG. 8 illustrates a process flow chart according to an alternative embodiment of the present invention in which extraction occurs at DDGS.

FIG. 9 illustrates a process flow chart of a five-stage reboiling process according to an embodiment of the present invention.

FIG. 10 illustrates a process flow chart of energy production from deoiled DWG according to an embodiment of the present invention.

FIG. 11 illustrates a process flow chart of a gasification process according to an embodiment of the present invention.

FIG. 12 is a table illustrating a comparison of alkyl acetate solvents used for extraction at varying temperatures for alternative ethanol production co-products.

### DESCRIPTION OF THE INVENTION

The invention is generally directed to a corn oil extraction process. The process includes the recovery of corn oil and other co-products, including but not limited to steam, electric power and chemicals from ethanol production process(es) and in particular, processes that involve dry corn milling methods. Generally, the process involves extraction of oil from milled corn and/or from residues from the fermentation step, including, but not limited to TS, DWG, DDG and DDGS and use of a food-grade solvent, such as but not limited to an alkyl acetate. These methods may be applied in existing facilities as retrofits, or may be applied in stand alone new ethanol plant construction.

To remove oil from still residues, the solvent employed preferably comprises an intrinsic hydrophobicity and includes properties making the solvent suitable for environmental, safety and health considerations. The solvent also preferably boils at a temperature favorable for its intended application and in particular, is acceptable for use in association with the ethanol production process and temperature ranges therein. More specifically, the solvent employed is capable of use at alternate stages of oil extraction associated with the processes described herein and more preferably between 60° C. and 99° C. and most preferably between 70° C. and 80° C., the maintained temperature of the stillage. The solvent further comprises a low solubility in water. Additionally, water also has low solubility in the solvent employed. The preferred solvent used for extraction is an ester and more preferably, an alkyl acetate and most preferably an alkyl acetate azeotrope. Exemplary alkyl acetates suitable for use as an extraction solvent include ethyl acetate, isopropyl acetate and butyl acetate and more preferably, ethyl acetate, although additional acetates and solvent compositions are contemplated. These solvents are currently commercially available from Celanese Corporation (Dallas, Tex.). In the preferred embodiment, ethyl acetate forms an azeotrope at approximately 91.8 wt % ethyl acetate and 8.2 wt % water, boiling at 70.4° C. These suitable solvents; ethyl acetate, isopropyl acetate and butyl acetate are produced from the esterification of acetic acid with ethanol, isopropanol and butanol respectively. Alternative solvents having any one or more of the foregoing properties would also be acceptable for the purposes of the present invention.

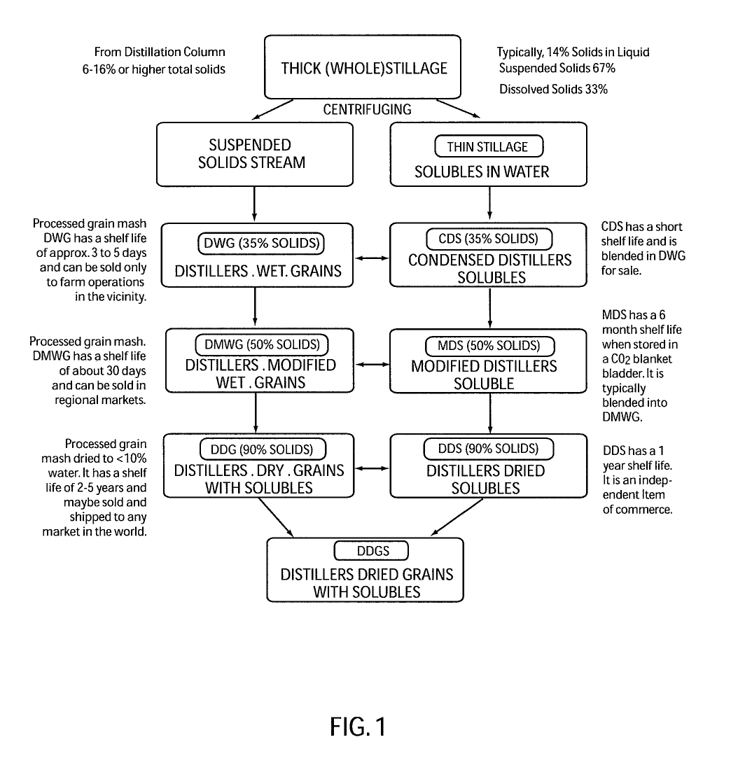

FIG. 1 represents a chart showing a traditional process flow in the ethanol production process, post fermentation. The process includes the production of common co-products of the ethanol production process, namely, DWG, DMWG, DDG, DDS and DDGS, all of which can be produced from the TS stream exiting the distillation column of the ethanol production facility.

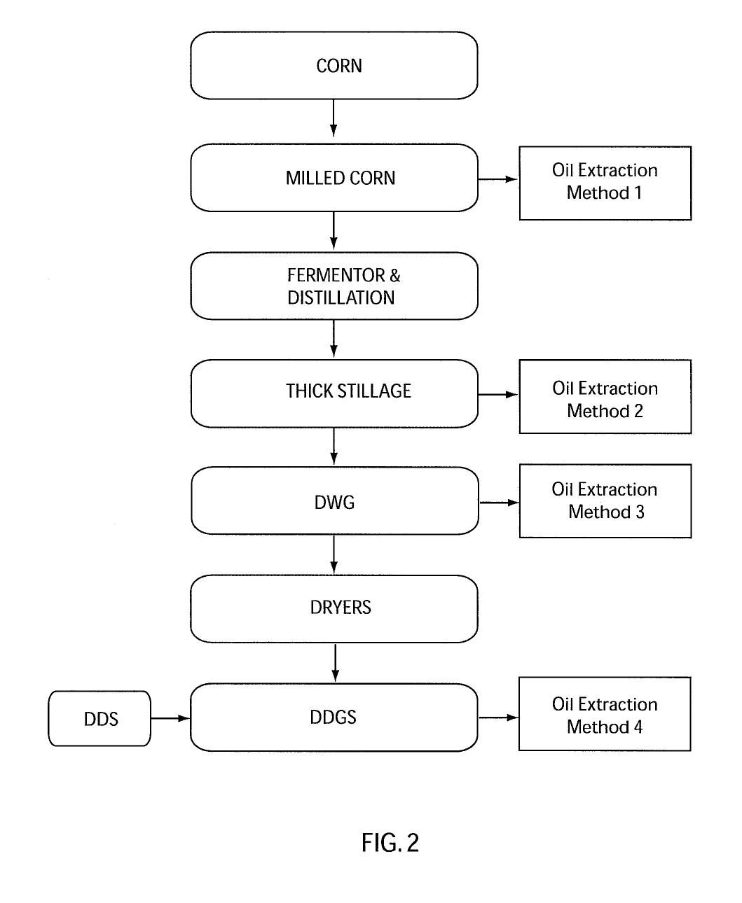

Generally, as shown in FIG. 2, the extraction step may occur in two locations of an ethanol production process. The process may occur upstream of the fermentors (method 1) or downstream, following fermentation. Application of the extraction step may occur at three alternative stages of the downstream process. In one embodiment, the extraction process may be applied to the TS (method 2). In an alternative embodiment, the extraction process may be applied to the DWG (method 3). In a further alternative embodiment, the extraction process may be applied to the DDGS (method 4). Many of the byproducts produced by one or more of the processes described herein may be recycled into the ethanol production process and corn oil extraction process. The methods described herein are numbered for purposes of ease of reference only. The methods and numbering thereof are not intended to be arranged in any particular order. One of skill in the art would understand that alternative method numbering, additional methods and/or combinations of the various methods described herein would not depart from the overall scope of the present invention.

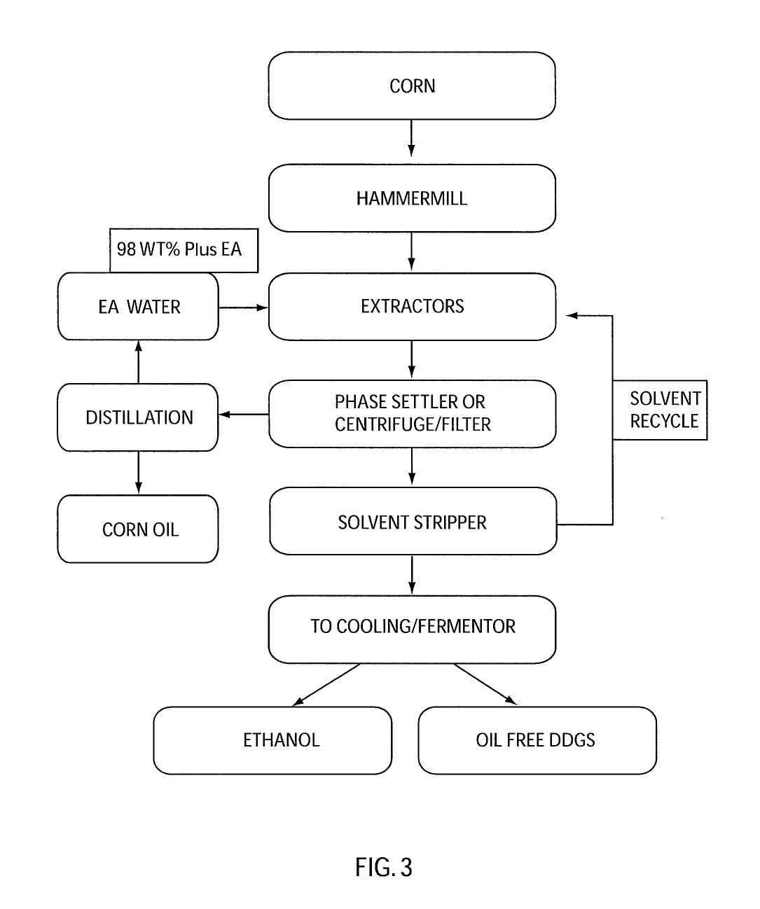

More specifically, in the embodiment illustrated in FIG. 3, extraction occurs prior to fermentation in the ethanol production process. In other words, in this “front end” approach, oil is extracted upstream of the fermentors.

In this embodiment, in further detail, the whole corn is initially transferred from corn storage silos into a hammermill. The hammermill grinds the whole corn to the required particle size, which is preferably provided having the following size distribution:

| MESH NO. | WT % |
|---|---|
| 16 | 5.44 |
| 20 | 9.99 |
| 30 | 15.74 |
| 40 | 17.35 |
| 60 | 23.42 |
| Bottom | 28.06 |

Alternative particle sizes would not depart from the overall scope of the present invention. Following grinding of the whole corn, the milled corn is transferred to the extractors. Preferably, the milled corn is transferred via commercially available conveyor systems linking the extractors with the hammermill. The hammermill and extractors are common commercially available equipment in the ethanol industry or may be custom built for the particular application. In the extractors, solvent is added or applied to the milled corn. Preferably, the solvent added includes fresh makeup solvent plus recycled solvent obtained from the process, such as from the solvent stripper, although variations or lack of such blend are also contemplated. The solvent or solvents are intensely blended with the milled corn to dissolve the corn oil content into the solvent. In one embodiment, the milled corn can be contacted with the solvent during the transfer process. By the time the milled corn reaches the extraction area, the solvent and milled corn have been in contact for a sufficient period of time such that the oils are removed from the milled corn and extraction can be undertaken without further delay. Generally this transfer process takes from about 2 to about 15 minutes, in particular from about 5 to 10 minutes and in particular about 3 to about 7 minutes. Blending may be conducted in any way commonly practiced in the art. For example, blending may involve use of a stirred tank vessel where intense mixing is generated by using an agitator. Alternately, blending may be conducted by using centrifugal agitators followed, in some instances, by a static soak tank. Following blending with the solvent and extraction of the oil, a separation step occurs in the phase settler which in one preferred embodiment separates the mixture into two separate and discreet phases. The top phase, including the dissolved corn oil in solvent, is pumped into a simple distillation column in which the solvent and water are removed, leaving corn oil as the bottom product. The separation step may also occur either through filtration or through centrifugation. The separation step removes the milled corn solids from the solvent, oil and water which are in the solvent phase. Acceptable separation systems include belt filters, rotary filters, centrifuges and other liquid solid separation equipment. Following separation of the milled corn solids from the solvent phase mixture, the oil can be separated from the mixture by simple distillation. In a preferred embodiment, distillation occurs by using a distillation column. The distillation column is generally equipped with a number of mass transfer stages with the preferred embodiment being either a tray-type or a packed column-type distillation column. The water and solvent are recovered from the top of the column and then recycled back into the process, returning the solvent/water mixture to the extractors.

Preferably, the water in solvent phase exists as an azeotrope. Optionally, the solvent can be dewatered although it is not necessary. In one embodiment, the water in solvent phase, using an alkyl acetate, such as but not limited to ethyl acetate, as solvent, contains 91.8 wt % alkyl acetate and 8.2 wt % water. Water content greater than 8.2 wt % in the solvent phase may be further separated by passing through an additional solvent/water distillation column. The preferred distillation column used incorporates an appropriate number of mass transfer stages, with the stages being either a tray type or a packed column type as indicated herein above.

The deoiled milled corn solids in the bottom solid phase are subjected to a desolventizing step to remove solvent absorbed by the milled corn solids. Preferably, solvent is removed by a solvent stripper.

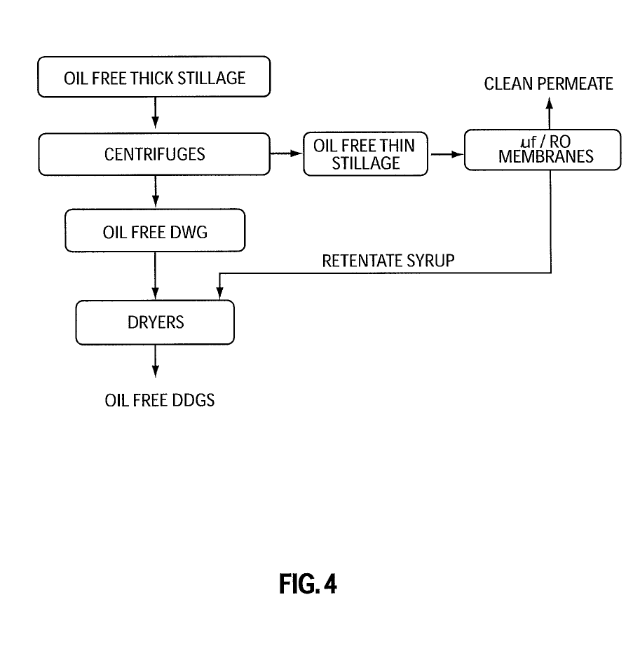

Once solvent is removed, the desolventized milled corn solids provide feedstock for the conventional dry corn milling ethanol production process. This feedstock provides significant advantages, as the downstream process is simplified over traditional methods. For example, as illustrated in FIG. 4, the TS may be placed in a centrifuge to separate the mixture so as to produce oil free DWG and oil free thin stillage. Namely, the TS emanating from the bottoms of the beer column is oil free and is passed through the centrifuges where the oil free DWG is routed to the dryers to dry. The oil free thin stillage may be filtered using membrane, such as, but not limited to a filtration membrane, including, but not limited to a micro-filtration membrane and/or reverse osmosis membrane. Acceptable membranes are available from Koch Industries (Wichita, Kans.), Siemens Corporation (New York, N.Y.), GE Osmonics (Minnetonka, Minn.). Filtration results in a clean permeate water stream and a retentate syrup. The retentate stream from this operation is a concentrate of proteinaceous and bacterial matter and may be directed into the dryers for co-blending with DWG as feed to yield an oil free DDGS product. Drying in a dryer includes application of steam and/or direct heated carbon dioxide and preferably, indirect application thereof, to produce oil free DDGS.

The thermal energy usage for a commercial dry milled corn ethanol plant is approximately 34,000 Btu/denatured gallon of ethanol produced. In comparison, the ethanol plant utilizing the foregoing process significantly reduces the thermal energy requirements to approximately 23,000 to 24,000 Btu and more preferably, to approximately 23,060 Btu/denatured gallon of ethanol produced. In addition, the pre-fermentation removal of oil reduces fermentation time, saving time and energy. Specifically, by removal of oil from the milled corn product, the enzymes operate more efficiently by working only on the remaining product.

In an alternative embodiment of the downstream, or post fermentation, method of extraction (method 2), oil is removed from the non-volatile residues that are pumped out and preferably continuously pumped out of the still. As discussed, the spent residue from the post fermentation distillation process is commonly referred to as TS and includes several major co-products. Typically, the TS contains between 10 wt % and 20 wt % solids and more preferably, about 14 wt % solids, both soluble and insoluble. This stream typically exits the still at a temperature ranging from 80° C. to 100° C., or more preferably, between 85° C. and 95° C. and most preferably, a range of 90° C. to 95° C.

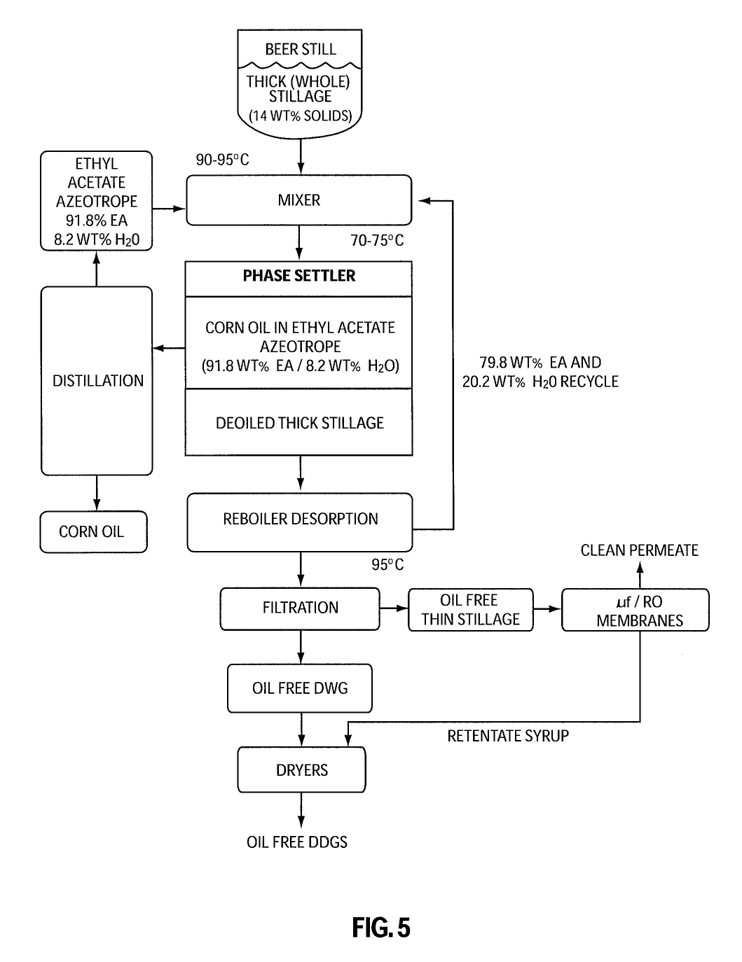

In the method shown in FIG. 5, corn oil is extracted from the TS produced downstream of the ethanol production facility. In one preferred embodiment, corn oil is extracted from the TS at the bottoms of the beer still. This also reduces the thermal requirement from 34,000 Btu to 23,060 Btu/denatured gallon of ethanol produced.

Typically, TS in the still typically exists at a temperature of 90° C. to 95° C. TS is cooled to a temperature of between 60° C. and 80° C. and preferably to a temperature between 65° C. and 75° C., and most preferably, a temperature range of from 70° C. to 75° C. The cooled TS is blended in a mixer, such as, but not limited to, a conventional stirred tank unit designed to afford an appropriate hold time and/or a centrifugal pump around loop, with a solvent and preferably, an alkyl acetate solvent and more preferably, an alkyl acetate azeotrope. Preferably, the solvent comprises a mixture of an alkyl acetate solvent stream, an alkyl acetate azeotrope mixture from a recycle stream of the ethanol production process (for instance, an ethyl acetate azeotrope containing approximately 91.8 wt % ethyl acetate and 8.2 wt % water) and a recycle stream recovered from a reboiler (containing approximately 79.8 wt % ethyl acetate and 20.2 wt % water). The recycle stream from the reboiler comprises a smaller stream than that received from the azeotrope recycle stream. The combined streams are intensely blended with the TS to dissolve the corn oil into the solvent.

The blended stream, including the solvent mixture and the TS, is pumped into a phase settler. In the phase settler the liquids split into two separate and discrete phases. A preferred phase settler is a common horizontally configured unit with an appropriate residence time. In some instances, the residence time can be decreased significantly by using electrostatic devices such as those available from NATCO (Houston, Tex.). The phase settler separates the corn oil in solvent from the deoiled TS, which separates or settles to the bottom of the phase settler. Namely, as a result of the hydrophobic nature of the alkyl acetate solvent, the liquids separate into two separate and discrete phases. In the preferred embodiment, the oil remains in the solvent phase, while protein remains in the water-solid phase. Thus, the top phase includes dissolved corn oil in solvent. The bottom phase includes deoiled TS in water-solid phase. The top phase is pumped into a distillation column and preferably, a simple elementary distillation column, where solvent is removed, leaving corn oil as the bottoms product. More specifically, the solvent, including alkyl acetate azeotrope, is boiled out of the solution in the distillation column, leaving corn oil as the resulting product. An acceptable distillation column is one with an appropriate number of mass transfer stages which may range from two (2) through twenty (20) and include either trays or packing available from Koch-Glitsch (related to Koch Industries of Wichita, Kans.). The number of mass transfer stages is contingent upon maintaining the desired purity of the recycle-stream.

The lower phase in the settler unit comprises primarily water and solids with a small amount of ethyl acetate. The solids included in the lower phase include both suspended and dissolved solids from the original TS. The lower phase is decanted into a reboiler desorption unit in which the solvent is stripped out and recycled back into the mixer. An acceptable reboiler desorption unit is a stripping column, typically with disc and donut trays offered by Koch-Glitsch (related to Koch Industries of Wichita, Kans.). Preferably, the lower phase mixture is heated to a temperature of between 70° C. and 110° C. and more preferably, between 80° C. and 105° C., preferably, to a range of from 90° C. to 100° C. and most preferably approximately 99° C. At this temperature, solvent, or more preferably, the alkyl acetate, is desorbed as a mixture of alkyl acetate and water, forming a mixture that comprises between 70 wt % and 90 wt % alkyl acetate and between 10 wt % and 30 wt % water and more preferably, between 75 wt % and 85 wt % alkyl acetate and 15 wt % and 25 wt % water and most preferably, approximately 79.8 wt % alkyl acetate and 20.2 wt % water. The desorbed mixture or stream is preferably recycled back into the mixer and applied to the TS as described hereinabove. In the preferred embodiment, the remaining stream of water from the desorption process will contain a minimal amount of solvent and preferably, less than 10 parts per million (ppm) of solvent, keeping solvent loss to a minimum.

The deoiled TS stream resulting from the foregoing process may be concentrated using a dewatering system, such as a filter press, rotary drum, belt, plate and frame, rotary press or other commercially available devices. A suitable dewatering device is a belt type filtration unit available from Larox Corporation (Lappeenranta, Finland). In a preferred embodiment, the dewatering device may be capable of optimization so as to yield a stream of solids which is approximately 35 wt % solids, having a remainder of water, protein and dissolved solids. Application of the dewatering device to the deoiled TS results in a deoiled filtrate (oil free thin stillage).

The deoiled filtrate may be further cleaned by passing through a membrane. An acceptable membrane includes units available from Koch, Siemens and GE Osmonics described hereinabove. Membranes may be selected in some embodiments which are suitable for various water needs, such as a pure water filter arrangement or in which water is needed or recycled back into the process. In the preferred embodiment, the water that passes through the filter is directed into a reverse osmosis unit. The clean water that exits the filter is recycled back into the fermentors as described above and the retentate, which is oil free backset, may be directed to a blending unit, for blending with the milled corn and introduction into a dryer. The retentate stream is preferably minimal and may be concentrated to yield a protein rich syrup or broth.

A wet filter cake of solids may also result from the filtration process. The wet filter cake will typically include residual moisture and often times include a significant amount of moisture. For example, a wet filter cake may contain between 60-70 wt % moisture.

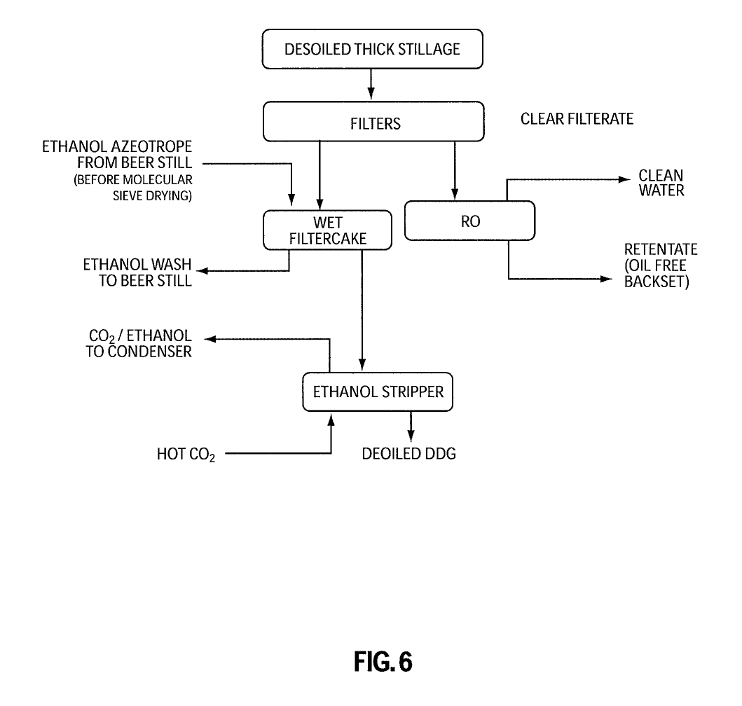

In one embodiment, as illustrated in FIG. 6, ethanol from the production process and more preferably, ethanol azeotrope formed from the overheads of the beer still may be used to wash the wet filter cake. The composition of solution used to wash the wet filter cake includes between 80 wt % and 100 wt % ethanol and more preferably, between 90 wt % and 100 wt % ethanol and most preferably approximately 95 wt % ethanol. This ethanol wash solution is preferably obtained from the still prior to molecular sieve drying. The ethanol laden wash stream is washed over the filter cake and dissolves the moisture remaining in the wet filter cake, resulting in a filter cake loaded with ethanol and a minimal amount of residual moisture, or water. The wet ethanol wash stream containing the moisture or water from the filter cake may be redirected into the beer still for separation. Thus, in the preferred embodiment, the moisture in the filter cake dissolves into the ethanol leaving the filter cake ethanol rich.

The ethanol rich filter cake is then dried. In the preferred embodiment, the cake is dried by the application of a stream of a gas having inert characteristics. CO₂ is a fermentation byproduct and is a readily available stream having inert characteristics. Preferably, the stream of inert gas is heated. Accordingly, in one preferred embodiment the process uses a heated stream of CO₂, which may be obtained from the fermentation process by recovering this stream downstream of the deodorant adsorbers. Preferably, the CO₂ stream is recovered by utilizing a pressurizing device which may be a recycle compressor/fan to move the CO₂ through dryers. The stream is preferably heated to a temperature above 70° C. and below 120° C. The stream preferably has a concentration of CO₂ ranging from 80 mole % to 100 mole %, which stream may include a diluent, such as water vapor and in some instances, a small amount of air which has occluded into the stream. The heated stream of CO₂ or inert gas applied to the ethanol rich filter cake desorbs the ethanol from the filter cake, yielding a deoiled filter cake or DDG product and a CO₂/ethanol stream. The ethanol/CO₂ stream may be routed to a conventional condenser in which the ethanol is removed as a liquid and the CO₂ recycled.

The filter cake from the presses or filtration system may be alternatively dewatered using conventional mechanical/thermal processing, such as, but not limited to, passing it through a rotary drum dryer, which may be directly or indirectly fired based upon energy optimization. In a preferred embodiment, conventional means may be applied to yield a stream containing about 70 wt % solids and 30 wt % water. The filter cake from the presses may be suitably dewatered by conventional means to yield an oil free DDG. Alternatively, the output stream may be optimized for feeding into a pressurized gasifier for the production of syngas. Optimization includes the consideration of atmospheric or pressurized dewatering. Often times, the stream may comprise a rich, heavy slurry. In this instance, it may be appropriate to use concrete pumps, such as a Putzmeister for pumping the stream. Gasifiers suitable for use include moving grate-types of units available from KMW Systems, Inc. (London, Ontario) and pressurized units like the type available from Carbona, Inc. (Helsinki, Finland). In some instances, a continuous gasification process will occur which will not need any lock hoppers and/or corresponding equipment. Syngas, as is known, can be used for a variety of purposes, including, but not limited to the production of ethanol and other products or co-products by known methods.

The corn oil generated using the foregoing method is acceptable for use in biodiesel production, or may be sent to a refining facility for additional product handling or refining, to sell the oil as a food grade material. Additionally, the ethanol-drying methodology described provides significant advantages, as it may eliminate conventional drying methodology and its incumbent heavy capital costs, energy costs and emission concerns. Moreover, the absence of oil in the filter cake eliminates the plugging problems of traditional systems that otherwise prevent use of reverse osmosis or micro-filtration membranes. This downstream process also appears to have the same capability as the first approach of reducing the thermal requirement of the ethanol facility to 23,060 Btu/denatured gallon of ethanol produced.

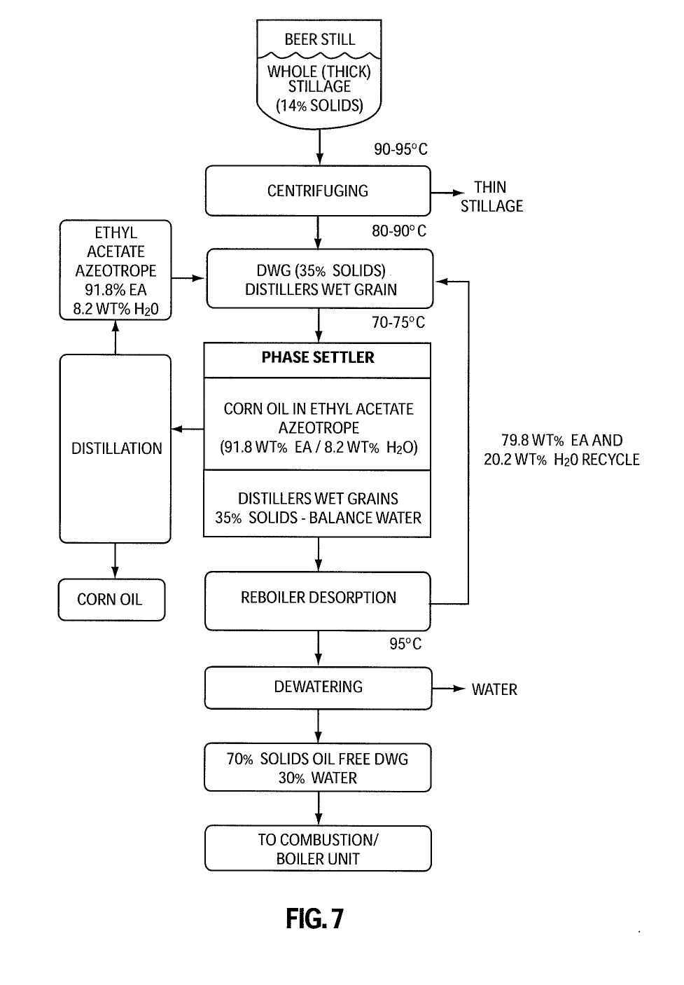

In method 3, or the second downstream method of corn oil extraction, shown in FIG. 7, oil is extracted from the DWG produced downstream of the ethanol production facility. In this embodiment, the TS is passed through a centrifugal liquid-solid separation device and is split into thin stillage and DWG. Suitable separation devices include centrifuges available from Flottweg AG (Vilsbiburg, Germany) and Westfalia Technologies, Inc. (York, Pa.). The separated DWG includes insoluble solids and water and more specifically a proportionate amount of water. Typically, the DWG stream includes 35 wt % solids in water. Centrifugation results in a temperature reduction of the DWG. Preferably, the temperature drops to a range of between 75° C. and 95° C. and more preferably a range of 80° C. to 90° C. The DWG stream is blended with solvent and preferably a three stream solvent similar to that described with the prior embodiment, including an alkyl acetate, such as ethyl acetate, an alkyl acetate azeotrope recycle stream (preferably having a concentration of 91.8 wt % ethyl acetate and 8.2 wt % water), plus a small recycle stream from the reboiler (preferably having a concentration of 79.8 wt % ethyl acetate and 20.2 wt % water). The small recycle stream is available from the recovery of the alkyl acetate which is dissolved in the process in a large quantity of water. The streams are intensely blended with DWG in a mixer to dissolve the corn oil in the solvent.

As with the previously described embodiment, the solvent stream having dissolved corn oil is pumped into a phase settler, which separates the stream into two separate and discreet phases. The top phase includes the dissolved corn oil in solvent. The top phase is, as discussed above, pumped into a simple distillation column in which the solvent is removed, leaving corn oil as the bottoms product. The lower phase in the settler, which includes solids in water, is as previously described, decanted into a reboiler desorption unit in which any remaining solvent is stripped out and recycled back into the mixer as described above.

The remaining DWG stream, free of corn oil, is dewatered as previously described or may be dewatered to 30 wt % water which is ideal for feeding into a pressurized gasifier for the production of syngas as was described for the TS above to produce the deoiled DWG having a moisture level of 30 wt %, the thermal energy requirement slightly reducing from 34,000 to 32,820 Btu/denatured gallon of ethanol.

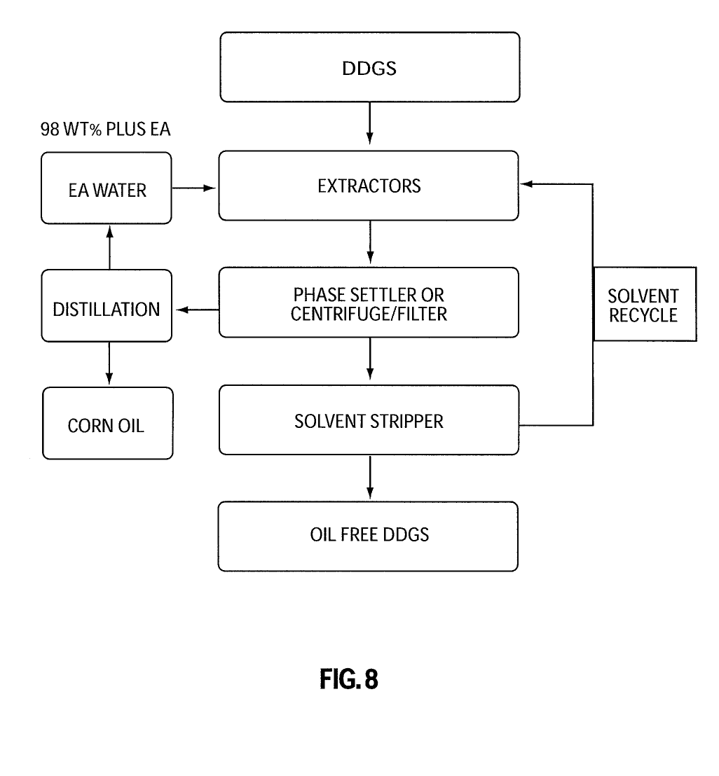

In method 4, or the third downstream method of corn oil extraction, oil is extracted from the final byproduct of the ethanol production process or drying process, namely DDGS, the dry solid residue. As illustrated in FIG. 8, similar to the corn oil extraction from milled corn using an alkyl acetate solvent, the DDGS is intensely blended with the solvent to dissolve the corn oil content in the DDGS into the solvent. The oil is then extracted in the same way as previously described for the corn oil extraction from milled corn (method 1).

The foregoing processes provide for significant improvement in the productivity of an ethanol production plant and more specifically may increase productivity by nearly 20 wt %. In addition, for each of the foregoing processes, the product remaining in each process, after the oil has been extracted, consists primarily of cellulosic and proteinaceous components that can be sold as animal feed. This deoiled material and residue also has value as fuel and can be used to raise steam and/or generate power, including but not limited to power for the production facility or other facilities. For example, for deoiled DDGS, namely, one (1) bushel of corn (56 lbs.) yields 16.8 lbs of deoiled DDGS having a fuel value of 6,900 Btu/lb and corn oil in DDGS having a fuel value of 1,500 Btu/lb. In addition, a food grade oil may be extracted using one or more of the foregoing methods. This oil can then be sold for food applications. Alternatively, the oil can be used as a feed stock for producing biodiesel. In addition, the deoiled milled corn and residues provide feedstock for the conventional dry corn milling ethanol plant where low energy filters are used in place of evaporators. The deoiled residues can also be gasified to product synthesis gas for ethanol, chemical and other power applications and they can also be pelletized for consumption as animal feed.

### EXAMPLES

The following Examples are set forth so as to provide those of ordinary skill in the art with a complete disclosure and description of the methods claimed herein, their performance and evaluation and are intended to be purely exemplary of the invention and are not intended to limit the scope of what is regarded as the invention. Efforts have been made to ensure accuracy with respect to numbers (e.g., amounts, temperature, etc.) but some errors and deviations should be taken into account.

### Example 1

The following analysis was performed related to oil extraction from TS or DWG.

TS and DWG samples were acquired from one corn dry mill ethanol plant nearby and were stored at −80° C. prior to usage.

Extraction of corn oil from TS or DWG was carried out using the assigned solvent and temperature based on the experimental designs. Triplicate extractions were conducted for each experimental condition. For each extraction, 25 mL of solvents were mixed with 2-10 g of TS or DWG. The extraction was conducted using a shaker for 1 hour, with a water bath to control the temperature. For each experiment, after the organic phase was separated, the residue was extracted for the second time using 15 mL of the same solvent for 30 minutes. The organic phases were combined for oil analysis. The solid residue was dried.

Corn oil content in TS or DWG was analyzed using AOAC (Association of Official Analytical Chemists) Official Method 945.16 (Petroleum Ether Extraction Method). Corn oil was extracted from TS or DWG with petroleum ether for 6 hours. The extract was filtered through small, hardened paper into a weighed vessel and then the paper was washed with a small portion of hot fresh solvent. After the solvent was evaporated at temperature of 70° C. and the dry vessel containing residue was dried in air in an oven for 1 hour at 100° C.-105° C., the weight of corn oil extracted was measured using a balance.

The content of water in TS or DWG was analyzed using AOAC Official Method 945.15 (Air Oven Method). The content of water in the solvent phase was analyzed following the method of Karl Fischer titration using a HYDRANAL moisture test kit purchased from Sigma. Gas chromatography coupled with flame ionization detection (GC-FID) was utilized for the analysis of the residue solvents after extraction. An Agilent 6890 gas chromatograph and a J&W Scientific 30-meter-long narrow-bore capillary column (DB5) with 0.25-μm phase thickness were utilized. The method applied for protein content analysis was Onishi & Barr Modified Lowry procedures using a test kit (Sigma TP 0200) purchased from Sigma.

Using ethyl acetate as the solvent, corn oil extraction experiments were conducted at three different temperatures: 35° C., 45° C. and 55° C. After extraction, the leftover solids were dried in an oven. Part of the solids was extracted again using petroleum ether to analyze the leftover oil and the other part was used for the analysis of protein content.

Since it is difficult to obtain a water phase in these experiments, the content of ethyl acetate in water was analyzed in this way: 50 mL of liquid was separated from TS through centrifugation and then the liquid was mixed with the same amount of ethyl acetate. Water phase samples were collected at three different temperatures: 35° C., 45° C. and 55° C. and analyzed using Gas Chromatography.

Using isopropyl acetate as the solvent, corn oil extraction experiments were conducted at six different temperatures: 45° C., 55° C., 65° C., 70° C., 80° C. and 90° C. After extraction, the leftover solids were dried in an oven. Part of the solids was extracted again using petroleum ether to analyze leftover oil and the other part was used for the analysis of protein content.

Since it is difficult to obtain a water phase in these experiments, the content of isopropyl acetate in water was analyzed in this way: 50 mL of liquid was separated from TS through centrifugation and then the liquid was mixed with the same amount of isopropyl acetate. Water phase samples were collected at six different temperatures: 45°, 55°, 65°, 70°, 80° and 90° C. and analyzed using Gas Chromatography.

### Example 2

### Corn Oil Extraction from TS Using Ethyl Acetate

The following represents one method of extracting oil from TS using ethyl acetate solvent. A total of 9 bench-scale tests were conducted at three different temperatures using ethyl acetate as the solvent. TS was intensely blended with solvent to perform the oil extraction. Experimentation and analysis was performed as set forth in Example 1. The results are shown in Tables 1, 2 and 3.

TABLE 1 summarizes the results of corn oil extraction from TS using ethyl acetate as solvent at 35° C. At 35° C., the Specific Gravity of ethyl acetate is 0.8848 and the content of ethyl acetate in water phase is 45.15 g/L. The amount of TS used in these tests is normalized to 10 g for comparison purposes.

TABLE 1
Results of Corn Oil Extraction from TS using
Ethyl Acetate as Solvent at 35° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| TS1 | 8.6963 | 0.1537 | 1.2505 | 24.59 | 0.12 | 27.5 |
| TS2 | 9.4857 | 0.1611 | 1.3498 | 25.02 | N/A | 27.8 |
| TS3 | 10.7799 | 0.1760 | 1.5437 | 24.73 | N/A | 27.1 |
| Average | 9.6540 | 0.1636 | 1.3814 | 24.78 | 0.12 | 27.5 |
| Normalized | 10.0000 | 0.1695 | 1.4308 | 24.78 | 0.12 | 27.5 |
| to 10 g | | | | | | |

Normalized for 10.0000 g of TS which gives water content of 8.5692 g and DM content of 1.4308 g. The DM is further divided into oil of 0.0014+0.1695=0.1709 g, protein of 0.3935+0.0049=0.3984 g and other DM of 0.8615 g. The combined extraction solution was allowed to settle into two phases. The solvent phase includes 41.05 ml or 36.3195 g ethyl acetate, 24.78 g/L×41.05 ml=1.0172 g water, 0.12 g/L×41.05 ml=0.0049 g protein and 0.1695 g oil, for a total of 37.5111 g, or 1.1916 g on an ethyl acetate free basis. The Water-solid phase includes water equal to 8.5692−1.0172=7.5520 g, oil equal to 0.0014 g, protein equal to 27.5 wt %×1.4308=0.3935 g, other DM equal to 0.8615 g and ethyl acetate equal to 45.15 g/L×7.5520 ml=0.3410 g=0.3854 ml, for a total of 9.1494 g, or 1.5974 g on a water free basis.

TABLE 2 summarizes the results of corn oil extraction from TS using ethyl acetate as solvent at a higher temperature, namely 45° C. At 45° C., the Specific Gravity of ethyl acetate is 0.8733 and the content of ethyl acetate in water phase is 48.21 g/L. The amount of TS used in these tests is normalized to 10 g for comparison purposes.

TABLE 2
Results of Corn Oil Extraction from TS using
Ethyl Acetate as Solvent at 45° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| TS1 | 6.5279 | 0.1110 | 0.9387 | 26.21 | 0.12 | 27.7 |
| TS2 | 7.6276 | 0.1369 | 1.0854 | 26.14 | N/A | 27.2 |
| TS3 | 9.3904 | 0.1516 | 1.3447 | 26.16 | N/A | 27.3 |
| Average | 7.8486 | 0.1332 | 1.1229 | 26.17 | 0.12 | 27.4 |
| Normalized | 10.0000 | 0.1697 | 1.4307 | 26.17 | 0.12 | 27.4 |
| to 10 g | | | | | | |

Normalized for 10.0000 g of TS which gives water content of 8.5693 g and DM content of 1.4307 g. The DM is further divided into oil of 0.0014+0.1697=0.1711 g, protein of 0.3920+0.0061=0.3981 g and other DM of 0.8615 g. The combined extraction solution was allowed to settle into two phases. The solvent phase includes 50.56 ml or 44.1540 g ethyl acetate, 26.17 g/L×50.56 ml=1.3232 g water, 0.12 g/L×50.56 ml=0.0061 g protein and 0.1697 g oil, for a total of 45.6530 g, or 1.4990 g on an ethyl acetate free basis. The water-solid phase includes water equal to 8.5693−1.3232=7.2461 g, oil equal to 0.0014 g, protein equal to 27.4 wt %×1.4307=0.3920 g, other DM equal to 0.8615 g and ethyl acetate equal to 48.21 g/L×7.2461 ml=0.3493 g=0.4000 ml, for a total of 8.8503 g, or 1.6042 g on a water free basis.

TABLE 3 summarizes the results of corn oil extraction from TS using ethyl acetate as solvent at a higher temperature, namely 55° C. At 55° C., the Specific Gravity of ethyl acetate is 0.8613 and the content of ethyl acetate in water phase is 51.18 g/L. The amount of TS used in these tests is normalized to 10 g for comparison purposes.

TABLE 3
Results of Corn Oil Extraction from TS using
Ethyl Acetate as Solvent at 55° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| TS1 | 8.6939 | 0.1522 | 1.2502 | 27.08 | 0.13 | 27.5 |
| TS2 | 8.4482 | 0.1354 | 1.2022 | 26.89 | N/A | 27.2 |
| TS3 | 9.4122 | 0.1680 | 1.3478 | 27.11 | N/A | 27.0 |
| Average | 8.8514 | 0.1519 | 1.2667 | 27.03 | 0.13 | 27.2 |
| Normalized | 10.0000 | 0.1716 | 1.4311 | 27.03 | 0.13 | 27.2 |
| to 10 g | | | | | | |

Normalized for 10.0000 g of TS which gives water content of 8.5689 g and DM content of 1.4311 g. The DM is further divided into oil of 0.0014+0.1716=0.1730 g, protein of 0.3893+0.0058=0.3993 g and other DM of 0.8588 g. The combined extraction solution was allowed to settle into two phases. The solvent phase includes 44.75 ml or 38.5437 g ethyl acetate, 27.03 g/L×44.75 ml=1.2096 g water, 0.13 g/L×44.75 ml=0.0058 g protein and 0.1716 g oil, for a total of 39.9307 g, or 1.3870 g on an ethyl acetate free basis. The water-solid phase includes water equal to 8.5689−1.2096=7.3593 g, oil equal to 0.0014 g, protein equal to 27.2 wt %×1.4311=0.3893 g, other DM equal to 0.8588 g and ethyl acetate equal to 51.18 g/L×7.3593 ml=0.3766 g=0.4372 ml, for a total of 8.9854 g, or 1.6261 g on a water free basis.

The foregoing results demonstrate that better than 99 wt % of the corn oil can be extracted from TS at varying temperatures. Secondly, the extraction results in a majority, or not all, the oil in the solvent phase and protein remaining in the water-solid phase. Third, temperature changes show limited or no effect on the strength of ethyl acetate extraction capability.

### Example 3

### Filtration of Two Phase Stillage

As indicated, the extraction solution separates into two phases. A total of 8 lab-scale filtration tests were conducted using a plate and frame type filter press, to filter the solids. A 70 wt % rubbing alcohol wash was used to wash the filtrate. TABLE 4 summarizes the filtration results for filtration tests 3-6 as examples. Experimentation and analysis was performed as set forth in Example 1.

TABLE 4
Summary of Filtration Test #3 Through #6

| Filtration Capacity | 4.0 to 9.2 kg/DS/m²/h |
|---|---|
| Liquid Capacity | 194-401 l/m²/h |
| Cake Moisture | 65.1 |

Some general observations were obtained from the filtrations tests. Specifically, moisture levels of 65 wt % were achieved while using 70 wt % rubbing alcohol as a wash, such results will likely be improved using 100 wt % alcohol/ethanol. Secondly, two phase separation occurs in Mother Liquor. Third, the resulting filtrate is clear. It is noted that additional tests on a full scale test unit will likely yield improved results, due to various factors such as dual sided filtration and washing variables available on the particular unit employed.

### Example 4

### Corn Oil Extraction from DWG Using Ethyl Acetate

The following represents one method of extracting oil from DWG using ethyl acetate solvent. A total of 9 bench-scale tests were conducted at three different temperatures using ethyl acetate as the solvent. DWG was intensely blended with solvent to perform the oil extraction. Experimentation and analysis was performed as set forth in Example 1. The results are shown in Tables 5, 6 and 7.

TABLE 5 summarizes the results of corn oil extraction from DWG using ethyl acetate as solvent at a temperature of 35° C. At 35° C., the Specific Gravity of ethyl acetate is 0.8848 and the content of ethyl acetate in water phase is 45.15 g/L. The amount of DWG used in these tests is normalized to 10 g for comparison purposes.

TABLE 5
Results of Corn Oil Extraction from DWG using Ethyl Acetate as
Solvent at 35° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| DWG1 | 3.3578 | 0.0734 | 1.0668 | 24.78 | 0.10 | 24.9 |
| DWG2* | 3.4081 | 0.0801 | 1.0511 | 24.66 | N/A | 25.2 |
| DWG3* | 3.1535 | 0.0759 | 0.9921 | 24.82 | N/A | 24.8 |
| Normalized | 10.0000 | 0.2186 | 3.0871 | 24.78 | 0.10 | 24.9 |
| to 10 g | | | | | | |

*not used in normalization

Normalized for 10.0000 g of DWG which gives water content of 6.9129 g and DM of 3.0871 g. The DM is further divided into oil of 0.0031+0.2186=0.2217 g, protein of 0.7687+0.0119=0.7806 g and other DM of 2.0848 g. The combined extraction solution was allowed to settle into two phases. The solvent phase includes 118.93 ml or 105.2293 g ethyl acetate, 24.78 g/L×118.93 ml=2.9471 g water, 0.10 g/L×118.93 ml=0.0119 g protein and 0.2186 g oil, for a total of 108.4069 g, or 3.1776 g on an ethyl acetate free basis. The water-solid phase includes water equal to 6.9129-2.9471=3.9658 g, oil equal to 0.0031 g, protein equal to 24.9 wt %×3.0871=0.7687 g, other DM equal to 2.0848 g and ethyl acetate equal to 45.15 g/L×3.9658 ml=0.1791 g=0.2024 ml, for a total of 6.2328 g, or 2.2670 g on a water free basis.

TABLE 6 summarizes the results of corn oil extraction from DWG using ethyl acetate as solvent at a higher temperature and in particular a temperature of 45° C. At 45° C., the Specific Gravity of ethyl acetate is 0.8733 and the content of ethyl acetate in water phase is 48.21 g/L. The amount of DWG used in these tests is normalized to 10 g for comparison purposes.

TABLE 6
Results of Corn Oil Extraction from DWG using Ethyl Acetate as
Solvent at 45° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| DWG1 | 2.4359 | 0.0498 | 0.7739 | 26.20 | 0.10 | 25.10 |
| DWG2 | 2.2987 | 0.0490 | 0.7089 | 26.05 | N/A | 24.80 |
| DWG3 | 2.7657 | 0.0621 | 0.8701 | 26.09 | N/A | 25.00 |
| Average | 2.5001 | 0.0536 | 0.7843 | 26.11 | 0.10 | 24.97 |
| Normalized | 10.0000 | 0.2144 | 3.1371 | 26.11 | 0.10 | 24.97 |
| to 10 g | | | | | | |

Normalized for 10.0000 g of DWG which gives the water content of 6.8629 g and DM of 3.1371 g. The DM is further divided into oil of 0.0031+0.2144=0.2175 g, protein of 0.7833+0.0160=0.7993 g and other DM of 2.1203 g. The combined extraction solution was allowed to settle into two phases. The solvent phase includes 159.84 ml or 139.5883 g ethyl acetate, 26.11 g/L×159.84 ml=4.1734 g water, 0.10 g/L×159.84 ml=0.0160 g protein and 0.2144 g oil, for a total of 143.9921 g, or 4.4038 g on an ethyl acetate free basis. The water-solid phase includes water equal to 6.8629−4.1734=2.6895 g, oil equal to 0.0031 g, protein equal to 24.97 wt %×3.1371=0.7833 g, other DM equal to 2.1203 g and ethyl acetate equal to 48.21 g/L×2.6895 ml=0.1297 g=0.1486 ml, for a total of 5.7259 g, or 3.0364 g on a water free basis.

TABLE 7 summarizes the results of corn oil extraction from DWG using ethyl acetate as solvent at a higher temperature and in particular a temperature of 55° C. At 55° C., the Specific Gravity of ethyl acetate is 0.8613 and the content of ethyl acetate in water phase is 51.18 g/L. The amount of DWG used in these tests is normalized to 10 g for comparison purposes.

TABLE 7
Results of Corn Oil Extraction from DWG using Ethyl Acetate as
Solvent at 55° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| DWG1 | 2.4801 | 0.0512 | 0.7879 | 27.03 | 0.12 | 24.6 |
| DWG2 | 2.4620 | 0.0542 | 0.7593 | 27.07 | N/A | 25.2 |
| DWG3 | 3.8099 | 0.0848 | 1.1986 | 27.12 | N/A | 24.9 |
| Average | 2.9173 | 0.0634 | 0.9153 | 27.07 | 0.12 | 24.9 |
| Normalized | 10.0000 | 0.2173 | 3.1375 | 27.07 | 0.12 | 24.9 |
| to 10 g | | | | | | |

Normalized for 10.0000 g of DWG which gives water content of 6.8625 g and DM of 3.1375 g. The DM is further divided into oil of 0.0031+0.2173=0.2204 g, protein of 0.7812+0.0164=0.7976 g and other DM of 2.1195 g. The combined extraction solution was allowed to settle into two phases. The solvent phase includes 136.92 ml or 117.9292 g ethyl acetate, 27.07 g/L×136.92 ml=3.7064 g water, 0.12 g/L×136.92 ml=0.0164 g protein and 0.2173 g oil, for a total of 121.8693 g, or 3.9401 g on an ethyl acetate free basis. The water-solid phase includes water equal to 6.8625-3.7064=3.1561 g, oil equal to 0.0031 g, protein equal to 24.9 wt %×3.1375=0.7812 g, other DM equal to 2.1195 g and ethyl acetate equal to 51.18 g/L×3.1561 ml=0.1615 g=0.1875 ml, for a total of 6.2214 g, or 3.0653 g on a water free basis.

As indicated in the method, water phase samples were collected at three different temperatures: 35°, 45° and 55° C. and analyzed using Gas Chromatography. Ethyl acetate content in the water phase was 45.15 g/L at 35° C., 48.21 g/L at 45° C. and 51.18 g/L at 55° C.

TABLE 8
Results of Corn Oil Extraction Using Ethyl Acetate as Solvent

| | | | | Water in | Protein in | Protein in |
|---|---|---|---|---|---|---|
| | Wet | Oil | Oil Yield, | Solvent | Solvent | Solids, wt %, |
| | Weight, g | Extracted, g | wt %, DM | Phase, g/L | Phase, g/L | DM |
| T = 35° C. | | | | | | |
| TS1 | 8.6963 | 0.1537 | 12.3513 | 24.59 | 0.12 | 27.5 |
| TS2 | 9.4857 | 0.1611 | 11.8682 | 25.02 | NA* | 27.8 |
| TS3 | 10.7799 | 0.1760 | 11.4093 | 24.73 | NA | 27.1 |
| DWG1 | 3.3578 | 0.0734 | 6.9706 | 24.78 | 0.10 | 24.9 |
| DWG2 | 3.4081 | 0.0801 | 7.4943 | 24.66 | NA | 25.2 |
| DWG3 | 3.1535 | 0.0759 | 7.6752 | 24.82 | NA | 24.8 |
| T = 45° C. | | | | | | |
| TS1 | 6.5279 | 0.1110 | 11.8831 | 26.21 | 0.12 | 27.7 |
| TS2 | 7.6276 | 0.1369 | 12.5424 | 26.14 | NA | 27.2 |
| TS3 | 9.3904 | 0.1516 | 11.2823 | 26.16 | NA | 27.3 |
| DWG1 | 2.4359 | 0.0498 | 6.5192 | 26.20 | 0.10 | 25.1 |
| DWG2 | 2.2987 | 0.049 | 6.7980 | 26.05 | NA | 24.8 |
| DWG3 | 2.7657 | 0.0621 | 7.1602 | 26.09 | NA | 25.0 |
| T = 55° C. | | | | | | |
| TS1 | 8.6939 | 0.1522 | 12.2337 | 27.08 | 0.13 | 27.5 |
| TS2 | 8.4482 | 0.1354 | 11.2003 | 26.89 | NA | 27.2 |
| TS3 | 9.4122 | 0.168 | 12.4731 | 27.11 | NA | 27.0 |
| DWG1 | 2.4801 | 0.0512 | 6.58352 | 27.03 | 0.12 | 24.6 |
| DWG2 | 2.4620 | 0.0542 | 7.01981 | 27.07 | NA | 25.2 |
| DWG3 | 3.8099 | 0.0848 | 7.09742 | 27.12 | NA | 24.9 |

*Not Analyzed

Ethyl acetate is effective in extracting corn oil out of TS and DWG, as the leftover oil content in the solids after extraction was too low to be quantified (<0.1 wt % DM). Furthermore, extraction results in oil and protein being separated into two different phases. The content of protein in the solvent phase was also very low, only about 0.1 g/L. The influence of temperature on oil extraction was not significant. There was no significant change in water content in the solvent phase either.

### Example 5

### Isopropyl Acetate Extraction from TS

The following represents one method of extracting oil from TS using isopropyl acetate solvent. 18 bench-scale tests were conducted at six different temperatures using isopropyl acetate (IPA) as the solvent. TS was intensely blended with solvent to perform the oil extraction. Experimentation and analysis was performed as set forth in Example 1. For purposes of example, nine test results at three different temperatures are shown in Tables 9, 10 and 11.

TABLE 9 summarizes the results of corn oil extraction from TS using isopropyl acetate as solvent at a temperature of 45° C. At 45° C., the Specific Gravity of isopropyl acetate is 0.8475 and the content of isopropyl acetate in water phase is 27.86 g/L. The amount of TS used in these tests is normalized to 10 g for comparison purposes.

TABLE 9
Results of Corn Oil Extraction from TS using Isopropyl Acetate as
Solvent at 45° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| TS1 | 4.9386 | 0.0886 | 0.7102 | 23.25 | 0.13 | 26.9 |
| TS2 | 5.4607 | 0.0919 | 0.7771 | 23.11 | N/A | 27.3 |
| TS3 | 5.5273 | 0.0962 | 0.7915 | 23.37 | N/A | 27.6 |
| Average | 5.3089 | 0.0922 | 0.7596 | 23.24 | 0.13 | 27.3 |
| Normalized | 10.0000 | 0.1737 | 1.4308 | 23.24 | 0.13 | 27.3 |
| to 10 g | | | | | | |

Normalized for 10.0000 g of TS which gives water content of 8.5692 g and DM content of 1.4308 g. The DM is further divided into oil of 0.0014+0.1737=0.1751 g, protein of 0.3906+0.0098=0.4004 g and other DM of 0.8553 g. The combined extraction solution was allowed to settle into two phases. The solvent phase includes 75.12 ml or 63.6642 g isopropyl acetate, 23.24 g/L×75.12 ml=1.7458 g water, 0.13 g/L×75.12 ml=0.0098 g protein and 0.1737 g oil, for a total of 65.5935 g, or 1.9293 g on an isopropyl acetate free basis. The water-solid phase includes water equal to 8.5692-1.7458=6.8234 g, oil equal to 0.0014 g, protein equal to 27.3 wt %×1.4308=0.3906 g, other DM equal to 0.8553 g and isopropyl acetate equal to 27.86 g/L×6.8234 ml=0.901 g=0.2243 ml, for a total of 8.2608 g, or 1.4374 g on a water free basis.

TABLE 10 summarizes the results of corn oil extraction from TS using isopropyl acetate as solvent at a higher temperature, namely a temperature of 65° C. At 65° C., the Specific Gravity of isopropyl acetate is 0.8240 and the content of isopropyl acetate in water phase is 30.82 g/L. The amount of TS used in these tests is normalized to 10 g for comparison purposes.

TABLE 10
Results of Corn Oil Extraction from TS using Isopropyl Acetate as
Solvent at 65° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| TS1 | 6.6895 | 0.1148 | 0.9620 | 26.02 | 0.13 | 27.1 |
| TS2 | 6.4477 | 0.1114 | 0.9175 | 25.79 | N/A | 26.9 |
| TS3 | 5.4108 | 0.0898 | 0.7748 | 26.13 | N/A | 27.3 |
| Average | 6.1827 | 0.1053 | 0.8848 | 25.98 | 0.13 | 27.1 |
| Normalized | 10.0000 | 0.1703 | 1.4311 | 25.98 | 0.13 | 27.1 |
| to 10 g | | | | | | |

Normalized for 10.0000 g of TS which gives water content of 8.5689 g and DM of 1.4311 g. The DM is further divided into oil of 0.0014+0.1703=0.1717 g, protein of 0.3878+0.0084=0.3962 g and other DM of 0.8632 g. The combined extraction solution was allowed to settle into two phases. The solvent phase includes 64.44 ml or 53.0986 g isopropyl acetate, 25.98 g/L×64.44 ml=1.6742 g water, 0.13 g/L×64.44 ml=0.0084 g protein and 0.1703 g oil, for a total of 54.9515 g, or 1.8529 g on an isopropyl acetate free basis. The water-solid phase includes water equal to 8.5689−1.6742=6.8947 g, oil equal to 0.0014 g, protein equal to 27.1 wt %×1.4311=0.3878 g, other DM equal to 0.8632 g and isopropyl acetate equal to 30.82 g/L×6.8947 ml=0.2125 g=0.2578 ml, for a total of 8.3596 g, or 1.4649 g on a water free basis.

TABLE 11 summarizes the results of corn oil extraction from TS using isopropyl acetate as solvent at a higher temperature, namely a temperature of 80° C. At 80° C., the Specific Gravity of isopropyl acetate is 0.8058 and the content of isopropyl acetate in water phase is 33.21 g/L. The amount of TS used in these tests is normalized to 10 g for comparison purposes.

TABLE 11
Results of Corn Oil Extraction from TS using Isopropyl Acetate as
Solvent at 80° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| TS1 | 7.0079 | 0.1133 | 1.0077 | 29.13 | 0.15 | 27.0 |
| TS2 | 6.4712 | 0.1113 | 0.9209 | 29.42 | N/A | 26.8 |
| TS3 | 6.8291 | 0.1211 | 0.9779 | 29.19 | N/A | 27.4 |
| Average | 6.7698 | 0.1152 | 0.9688 | 29.25 | 0.15 | 27.1 |
| Normalized | 10.0000 | 0.1702 | 1.4311 | 29.25 | 0.15 | 27.1 |
| to 10 g | | | | | | |

Normalized for 10.0000 g of TS which gives water content of 8.5689 g and DM of 1.4311 g. The DM is further divided into oil of 0.0014+0.1702=0.1716 g, protein of 0.3878+0.0088=0.3966 g and other DM of 0.8629 g. The combined extraction solution was allowed to settle into two phases. The solvent phase includes 58.80 ml or 47.3810 g isopropyl acetate, 29.25 g/L×58.80 ml=1.7199 g water, 0.15 g/L×58.80 ml=0.0088 g protein and 0.1702 g oil, for a total of 49.2799 g, or 1.8989 g on an isopropyl acetate free basis. The water-solid phase includes water equal to 8.5689−1.7199=6.8490 g, oil equal to 0.0014 g, protein equal to 27.1 wt %×1.4311=0.3878 g, other DM equal to 0.8629 g and isopropyl acetate equal to 33.21 g/L×6.8490 ml=0.2275 g=0.2823 ml, for a total of 8.3286 g, or 1.4796 g on a water free basis.

From the foregoing, it is understood that, similar to ethyl acetate, isopropyl acetate is also effective in extracting corn oil from TS. Moreover, temperature has little or no effect on the effectiveness of isopropyl acetate solvent in removal of oil. The amount of isopropyl acetate in water phase is smaller in the above results than ethyl acetate due to the decreased solubility of isopropyl acetate in water.

### Example 6

### Corn Oil Extraction from DWG Using Isopropyl Acetate

The following represents one method of extracting oil from DWG using isopropyl acetate solvent. 18 bench-scale tests were conducted at six different temperatures using isopropyl acetate as the solvent. DWG was intensely blended with solvent to perform the oil extraction. Experimentation and analysis was performed as set forth in Example 1. As examples, nine test results at three different temperatures are shown in Tables 12, 13 and 14.

TABLE 12 summarizes the results of corn oil extraction from DWG using isopropyl acetate as solvent at a temperature of 45° C. At 45° C., the Specific Gravity of isopropyl acetate is 0.8475 and the content of isopropyl acetate in water phase is 27.86 g/L. The amount of DWG used in these tests is normalized to 10 g for comparison purposes.

TABLE 12
Results of Corn Oil Extraction from DWG using Isopropyl Acetate as
Solvent at 45° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| DWG1 | 2.3059 | 0.0461 | 0.7326 | 23.78 | 0.10 | 25.10 |
| DWG2 | 2.4705 | 0.0545 | 0.7619 | 23.61 | N/A | 24.60 |
| DWG3 | 2.1957 | 0.0492 | 0.6908 | 23.34 | N/A | 24.90 |
| Average | 2.3240 | 0.0499 | 0.7284 | 23.58 | 0.10 | 24.87 |
| Normalized | 10.0000 | 0.2147 | 3.1343 | 23.58 | 0.10 | 24.87 |
| to 10 g | | | | | | |

Normalized for 10.0000 g of DWG which gives water content of 6.8657 g and DM of 3.1343 g. The DM is further divided into oil of 0.0031+0.2147=0.2178 g, protein of 0.7795+0.0172=0.7967 g and other DM of 2.1198 g. The combined extraction solution was allowed to settle into two phases. The solvent phase includes 172.03 ml or 145.7954 g isopropyl acetate, 23.58 g/L×172.03 ml=4.0565 g water, 0.10 g/L×172.03 ml=0.0172 g protein and 0.2147 g oil, for a total of 150.0838 g, or 4.2884 g on an isopropyl acetate free basis. The water-solid phase includes water equal to 6.8657−4.0565=2.8092 g, oil equal to 0.0031 g, protein equal to 24.87 wt %×3.1343=0.7795 g, other DM equal to 2.1198 g and isopropyl acetate equal to 27.86 g/L×2.8092 ml=0.0783 g=0.0924 ml, for a total of 5.7899 g, or 2.9807 g on a water free basis.

TABLE 13 summarizes the results of corn oil extraction from DWG using isopropyl acetate as solvent at a higher temperature, namely a temperature of 65° C. At 65° C., the Specific Gravity of isopropyl acetate is 0.8240 and the content of isopropyl acetate in water phase is 31.56 g/L. The amount of DWG used in these tests is normalized to 10 g for comparison purposes.

TABLE 13
Results of Corn Oil Extraction from DWG using Isopropyl
Acetate as Solvent at 65° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| DWG1 | 2.4786 | 0.0512 | 0.7875 | 26.06 | 0.12 | 24.7 |
| DWG2 | 2.4558 | 0.0529 | 0.7574 | 25.91 | N/A | 25.1 |
| DWG3 | 3.8104 | 0.0841 | 1.1988 | 26.12 | N/A | 24.8 |
| Average | 2.9149 | 0.0627 | 0.9146 | 26.03 | 0.12 | 24.9 |
| Normalized | 10.0000 | 0.2151 | 3.1375 | 26.03 | 0.12 | 24.9 |
| to 10 g | | | | | | |

Normalized for 10.0000 g of DWG which gives water content of 6.8625 g and DM of 3.1375 g. The DM is further divided into oil of 0.0031+0.2151=0.2182 g, protein of 0.7812+0.0165=0.7977 g and other DM of 2.1216 g. The combined extraction solution was allowed to settle into two phases. The solvent phase includes 137.10 ml or 112.9703 g isopropyl acetate, 26.03 g/L×137.10 ml=3.5687 g water, 0.12 g/L×137.10 ml=0.0165 g protein and 0.2151 g oil, for a total of 116.7706 g, or 3.8003 g on an isopropyl acetate free basis. The water-solid phase includes water equal to 6.8625−3.5687=3.2938 g, oil equal to 0.0031 g, protein equal to 24.9 wt %×3.1375=0.7812 g, other DM equal to 2.1216 g and isopropyl acetate equal to 31.56 g/L×3.2938 ml=0.1039 g=0.1261 ml, for a total of 6.3036 g, or 3.0098 g on a water free basis.

TABLE 14 summarizes the results of corn oil extraction from DWG using isopropyl acetate as solvent at a higher temperature, namely a temperature of 80° C. At 80° C., the Specific Gravity of isopropyl acetate is 0.8058 and the content of isopropyl acetate in water phase is 33.21 g/L. The amount of DWG used in these tests is normalized to 10 g for comparison purposes.

TABLE 14
Results of Corn Oil Extraction from DWG using Isopropyl Acetate as
Solvent at 80° C.

| | | | | Water | Protein | Protein |
|---|---|---|---|---|---|---|
| | | | | in | in | in |
| | Wet | Oil | | Solvent | Solvent | Solids, |
| | Weight, | Extracted, | | Phase, | Phase, | wt %, |
| | g | g | DM, g | g/L | g/L | DM |
| DWG1 | 2.3313 | 0.0499 | 0.7407 | 29.21 | 0.13 | 24.9 |
| DWG2 | 2.0498 | 0.0474 | 0.6322 | 28.96 | N/A | 24.8 |
| DWG3 | 2.7838 | 0.0615 | 0.8758 | 29.07 | N/A | 25.3 |
| Average | 2.3883 | 0.0529 | 0.7495 | 29.08 | 0.13 | 25.0 |
| Normalized | 10.0000 | 0.2215 | 3.1382 | 29.08 | 0.13 | 25.0 |
| to 10 g | | | | | | |

Normalized for 10.0000 g of DWG which gives the water content of 6.8618 g and DM of 3.1382 g. The DM is further divided into oil of 0.0031+0.2215=0.2246 g, protein of 0.7846+0.0218=0.8064 g and other DM of 2.1072 g. The combined extraction solution was allowed to settle into two phases. The solvent phase includes 167.40 ml or 134.8909 g isopropyl acetate, 29.08 g/L×167.40 ml=4.8680 g water, 0.13 g/L×167.40 ml=0.0218 g protein and 0.2215 g oil, for a total of 140.0022 g, or 5.1113 g on an isopropyl acetate free basis. The water-solid phase includes water equal to 6.8618-4.8680=1.9938 g, oil equal to 0.0031 g, protein equal 25.0 wt %×3.1382=0.7846 g, other DM equal to 2.1072 g and isopropyl acetate equal to 33.21 g/L×1.9938 ml=0.0662 g=0.0822 ml, for a total of 4.9549 g, or 2.9611 g on a water free basis. As indicated in the method, water phase samples were collected at six different temperatures: 45°, 55°, 65°, 70°, 80° and 90° C. The Gas Chromatography results of these samples showed that isopropyl acetate content in the water phase was 27.86 g/L at 45° C., 29.31 g/L at 55° C., 30.82 g/L at 65° C., 31.56 g/L at 70° C., 33.21 g/L at 80° C. and 50.35 g/L at 90° C.

TABLE 15
Results of Corn Oil Extraction Using Isopropyl Acetate as Solvent

| | | | | Water in | Protein in | Protein in |
|---|---|---|---|---|---|---|
| | Wet | Oil | Oil Yield, | Solvent | Solvent | Solids, |
| | Weight, g | Extracted, g | wt %, DM | Phase, g/L | Phase, g/L | wt %, DM |
| T = 45° C. | | | | | | |
| TS1 | 4.9386 | 0.0886 | 12.5371 | 23.25 | 0.13 | 26.9 |
| TS2 | 5.4607 | 0.0919 | 11.7609 | 23.11 | NA* | 27.3 |
| TS3 | 5.5273 | 0.0962 | 12.1634 | 23.37 | NA | 27.6 |
| DWG1 | 2.3059 | 0.0461 | 6.3753 | 23.78 | 0.10 | 25.1 |
| DWG2 | 2.4705 | 0.0545 | 7.0349 | 23.61 | NA | 24.6 |
| DWG3 | 2.1957 | 0.0492 | 7.1460 | 23.34 | NA | 24.9 |
| T = 55° C. | | | | | | |
| TS1 | 8.2875 | 0.1364 | 11.5018 | 24.29 | 0.13 | 27.4 |
| TS2 | 6.1704 | 0.1072 | 12.1418 | 23.81 | NA | 26.8 |
| TS3 | 5.1213 | 0.0849 | 11.5841 | 24.16 | NA | 27.1 |
| DWG1 | 3.0825 | 0.0645 | 6.6721 | 24.08 | 0.11 | 25.0 |
| DWG2 | 3.1683 | 0.0688 | 6.9243 | 24.15 | NA | 24.5 |
| DWG3 | 2.3815 | 0.0514 | 6.8827 | 23.98 | NA | 25.2 |
| T = 65° C. | | | | | | |
| TS1 | 6.6895 | 0.1148 | 11.9920 | 26.02 | 0.13 | 27.1 |
| TS2 | 6.4477 | 0.1114 | 12.0732 | 25.79 | NA | 26.9 |
| TS3 | 5.4108 | 0.0898 | 11.5975 | 26.13 | NA | 27.3 |
| DWG1 | 2.4786 | 0.0512 | 6.5869 | 26.06 | 0.12 | 24.7 |
| DWG2 | 2.4558 | 0.0529 | 6.8692 | 25.91 | NA | 25.1 |
| DWG3 | 3.8104 | 0.0841 | 7.0383 | 26.12 | NA | 24.8 |
| T = 70° C. | | | | | | |
| TS1 | 5.978 | 0.1019 | 11.9118 | 27.91 | 0.13 | 27.6 |
| TS2 | 5.1124 | 0.0837 | 11.4409 | 27.24 | NA | 27.7 |
| TS3 | 6.3153 | 0.1116 | 12.3489 | 27.37 | NA | 27.2 |
| DWG1 | 2.0013 | 0.0393 | 6.2618 | 27.88 | 0.12 | 24.8 |
| DWG2 | 2.5419 | 0.0525 | 6.5860 | 27.65 | NA | 25.1 |
| DWG3 | 2.6141 | 0.0559 | 6.8188 | 27.81 | NA | 24.9 |
| T = 80° C. | | | | | | |
| TS1 | 7.0079 | 0.1133 | 11.2980 | 29.13 | 0.15 | 27.0 |
| TS2 | 6.4712 | 0.1113 | 12.0191 | 29.42 | NA | 26.8 |
| TS3 | 6.8291 | 0.1211 | 12.3919 | 29.19 | NA | 27.4 |
| DWG1 | 2.3313 | 0.0499 | 6.8253 | 29.21 | 0.13 | 24.9 |
| DWG2 | 2.0498 | 0.0474 | 7.3737 | 28.96 | NA | 24.8 |
| DWG3 | 2.7838 | 0.0615 | 7.0446 | 29.07 | NA | 25.3 |
| T = 90° C. | | | | | | |
| TS1 | 5.2885 | 0.0907 | 11.9849 | 52.01 | 0.15 | 26.9 |
| TS2 | 5.6949 | 0.1005 | 12.3322 | 51.85 | NA | 27.0 |
| TS3 | 7.7857 | 0.1262 | 11.3271 | 52.32 | NA | 27.3 |
| DWG1 | 2.0734 | 0.0419 | 6.4439 | 52.04 | 0.13 | 24.6 |
| DWG2 | 3.1944 | 0.0639 | 6.3787 | 52.07 | NA | 24.9 |
| DWG3 | 2.2790 | 0.0488 | 6.8280 | 52.16 | NA | 25.1 |

*Not Analyzed

From the foregoing, it is understood that, similar to ethyl acetate, isopropyl acetate is effective in extracting corn oil from DWG as well as TS, as the leftover oil content in the solids after extraction was too low to be quantified (<0.1 wt % DM) The content of protein in the solvent phase was also very low, only about 0.1 g/L. In addition, temperature has little or no effect on the effectiveness of the isopropyl acetate solvent in removal of oil. The amount of isopropyl acetate in water phase is smaller in the above results than ethyl acetate due to the decreased solubility of isopropyl acetate in water.

### Example 7

### Corn Oil Extraction from DDGS Using Ethyl Acetate

The following represents one method of extracting oil from DDGS using ethyl acetate solvent at 25° C. Bench-scale tests were conducted at three different solvent to DDGS ratios. DDGS was intensely blended with solvent to perform the oil extraction. Specifically, 8.6 g of DDGS was intensely blended respectively, with 10 ml, 20 ml and 30 ml solvent to arrive at the different ratios. Three extractions were performed for each ratio. Experimentation and analysis were performed similar as set forth in Example 1. The results are listed in TABLE 16.

TABLE 18 summarizes the results of corn oil extraction from DDGS using ethyl acetate as solvent at a temperature of 25° C. At 25° C., the Specific Gravity of ethyl acetate is 0.8963, the content of ethyl acetate in water phase is 42.09 g/L and water content in the solvent phase is 23.39 g/L. 8.6 g of DDGS was normalized to 10 g in these tests for comparison purposes.

TABLE 16
Results of Corn Oil Extraction from DDGS using
Ethyl Acetate as Solvent at 25° C.

| | | | |
|---|---|---|---|
| DDGS 8.6 g | 1:1 | 2:1 | 3:1 |
| Oil (g) | 10 ml Solvent each | 20 ml Solvent each | 30 ml |
| | | | Solvent each |
| 1^{st} extraction | 0.0723 | 0.2425 | 0.3440 |
| 2^{nd} extraction | 0.3086 | 0.2944 | 0.2809 |
| 3^{rd} extraction | 0.1934 | 0.1375 | 0.1003 |
| Total | 0.5743 | 0.6744 | 0.7252 |

### Normalized to 10 g of Corn:

| | | | |
|---|---|---|---|
| DDGS 10 g | 1:1 | 2:1 | 3:1 |
| Oil (g) | 11.63 ml Solvent | 23.26 ml Solvent | 34.88 ml Solvent |
| | each | each | each |
| 1^{st} extraction | 0.0841 | 0.2820 | 0.4000 |
| 2^{nd} extraction | 0.3588 | 0.3423 | 0.3266 |
| 3^{rd} extraction | 0.2249 | 0.1599 | 0.1166 |
| Total | 0.6678 | 0.7842 | 0.8432 |

Normalized for 10.0000 g of DDGS which gives water content of 1.0000 g and DM of 9.0000 g. The DM is further divided into oil of 0.9281 g, protein of 3.1270 g and other of 4.9449 g DM. The solvent used for extraction is 104.65 ml ethyl acetate. However, the maximum amount of water content in 104.65 ml ethyl acetate is 23.89 g/L×104.65 ml=2.4478 g at 25° C. which is much higher than 1.0000 g water. Therefore, there is no water phase present. This process results in 0.8432 g oil extracted and 0.0849 g oil remaining after the 3^{rd} extraction with 34.88 ml of solvent used for each extraction.

The oil content in the DDGS was measured to be 0.9281 g. Therefore, 90.85 wt % of the oil was extracted after the 3^{rd} extraction with 3:1 solvent to DDGS ratio. For higher oil recovery efficiency, both a 4^{th} extraction and higher solvent to DDGS ratio may be utilized due to the high oil content in DDGS.

### Example 8

### Corn Oil Extraction from Milled Corn Using Ethyl Acetate as Solvent

The following represents one method of extracting oil from milled corn using ethyl acetate solvent. Bench-scale tests were conducted at three different solvent to milled corn ratios and are listed in Table 17. 8.6 g of milled corn was intensely blended respectively, with 10 ml, 20 ml and 30 ml solvent to arrive at the different ratios. Three extractions were performed for each ratio. Experimentation and analysis were performed similar as set forth in Example 1.

TABLE 17 illustrates the results of corn oil extraction by use of ethyl acetate solvent at a temperature of 25° C. At 25° C., the Specific Gravity of ethyl acetate is 0.8963, the content of ethyl acetate in water phase is 42.09 g/L and the water content in solvent phase is 23.39 g/L.

TABLE 17
Results of Corn Oil Extraction from Milled Corn using Ethyl
Acetate as Solvent at 25° C.

| | | | |
|---|---|---|---|
| Corn 8.6 g | 1:1 | 2:1 | 3:1 |
| Oil (g) | 10 ml Solvent | 20 ml Solvent | 30 ml Solvent each |
| | each | each | |
| 1^{st} extraction | 0.1035 | 0.1612 | 0.2040 |
| 2^{nd} extraction | 0.0974 | 0.0921 | 0.0917 |
| 3^{rd} extraction | 0.0467 | 0.0315 | 0.0269 |
| Total | 0.2486 | 0.2848 | 0.3226 |

The 8.6 g of milled corn is normalized to 10 g in order to compare other corn oil extraction results from other tests. Normalized to 10 g of Corn:

| | | | |
|---|---|---|---|
| Corn 10 g | 1:1 | 2:1 | 3:1 |
| Oil (g) | 11.63 ml Solvent | 23.26 ml Solvent | 34.88 ml Solvent |
| | each | each | each |
| 1^{st} extraction | 0.1203 | 0.1874 | 0.2372 |
| 2^{nd} extraction | 0.1133 | 0.1071 | 0.1066 |
| 3^{rd} extraction | 0.0543 | 0.0366 | 0.0313 |
| Total | 0.2879 | 0.3311 | 0.3751 |

For 10.0000 g of milled corn, which gives water content of 1.5000 g and dry matter (DM) of 8.5000 g. The DM can be further divided into 0.3962 g oil, 0.8583 g protein and 7.2455 g other DM. The solvent used for extraction is 104.65 ml ethyl acetate. However, the maximum amount of water content in 104.65 ml ethyl acetate is 23.39 g/L×104.65 ml=2.4478 g at 25° C. which is much higher than 1.5000 g. Therefore, there is no water phase present. Oil extracted from the 3:1 mixture was 0.3751 g, with oil remaining of 0.0211 g.

The corn oil content in 10 g of milled corn is measured as 0.3962 g and the oil extracted from the 3:1 solvent to milled corn ratio after the 3^{rd} extraction is 0.3751 g. Therefore, 94.67 wt % of the oil was recovered after the 3^{rd} extraction with 3:1 solvent to milled corn ratio. For higher oil recovery efficiency, either a 4^{th} extraction or higher solvent to milled corn ratio may be required.

### Example 9

### Corn Oil Extraction from DWG Using Ethyl Acetate Solvent

When the DWG stream (35 wt % solids, balance primarily water) is blended with a solvent, ethyl acetate in this case, two phases form: (1) The upper phase comprises of corn oil in ethyl acetate which still contains a small amount of water; (2) The lower phase comprises primarily of water and solids (35 wt %) with a small amount of ethyl acetate—typically around 7 wt % at the temperatures discussed herein.

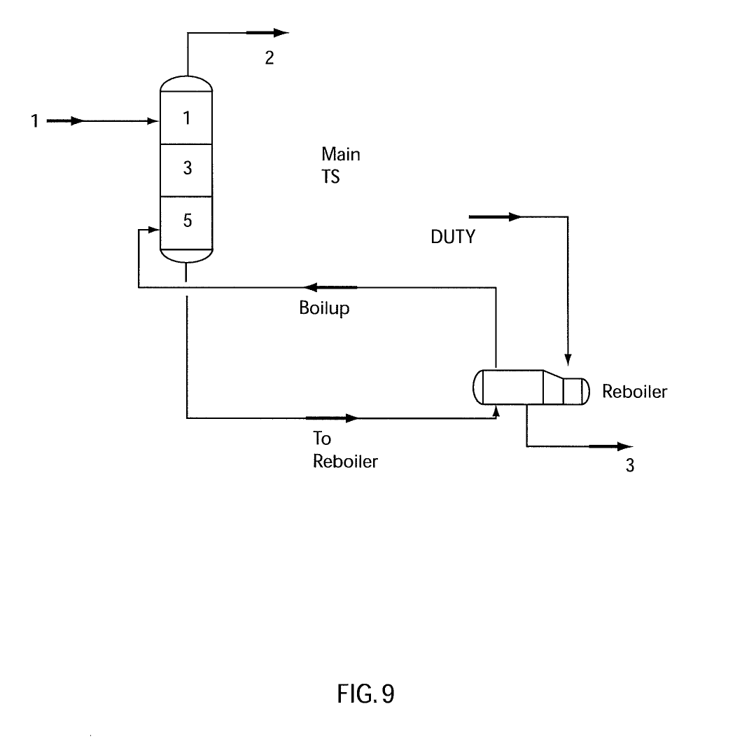

This lower phase is decanted, preheated to about 77° C. and then directed into a reboiler desorption unit. As shown in FIG. 9, a detailed process model is used to simulate the operation of the reboiler desorption unit for bench scale analysis. Five stages were used in the unit so that the ethyl acetate content in the third stream stays less than 1 ppm by weight. Ethyl acetate content of less than 1 ppm has significant advantages, as the ethyl acetate losses are kept to a minimum and no wastewater treatment is required.

The tables and diagram below show the simulated results of material streams, temperature profiles, net liquid rates and net vapor rates in the five-stage reboiler desorption unit.

TABLE 18
5-STAGE REBOILER DESORPTION UNIT PROFILES

| | Name of Stream | | | | |
|---|---|---|---|---|---|
| | 2 | To Reboiler | Boil up | 3 | 1 |
| Vapor Fraction | 1.000 | 0.00 | 1.00 | 0.00 | 0.00 |
| Temperature (° C.) | 83.590 | 99.41 | 99.41 | 99.41 | 77.00 |
| Pressure (psia) | 14.500 | 14.50 | 14.50 | 14.50 | 14.50 |
| Molar Flow (lbmole/hr) | 7.726 | 227.30 | 14.60 | 212.70 | 220.50 |
| Mass Flow (lb/hr) | 381.200 | 4,095.00 | 263.00 | 3,832.00 | 4,214.00 |
| Liquid Volume Flow | 28.300 | 281.00 | 18.04 | 262.90 | 291.20 |
| (barrel/day) | | | | | |
| Heat Flow (kJ/h) | −1.087e+006 | −2.730e+007 | −1.497e+006 | −2.555e+007 | −2.689e+007 |

| | | Pressure | | Net Liquid | Net Vapor |
|---|---|---|---|---|---|
| | Stage | (psia) | Temp (° C.) | (lb/mole/hr) | (lb/mole/hr) |
| 1_Main TS | 0 | 14.50 | 83.59 | 221.9 | 7.726 |
| 2_Main TS | 1 | 14.50 | 96.09 | 226.1 | 9.159 |
| 3_Main TS | 2 | 14.50 | 98.97 | 227.2 | 13.36 |
| 4_Main TS | 3 | 14.50 | 99.36 | 227.3 | 14.42 |
| 5_Main TS | 4 | 14.50 | 99.41 | 227.3 | 14.57 |
| Reboiler | 5 | 14.50 | 99.41 | 212.7 | 14.60 |

It should be noted that only a small boil up stream which accounts for 6.4 mole % (6.5 wt %) of the reboiler feed stream is needed to desorb out all the ethyl acetate in the reboiler desorption unit feed stream. As a result, the process requires a very small energy demand for the unit.

Stream 2 is preferably about 3.5 mole % (14.5 wt %) of the feed stream to the unit and preferably contains 79.8 wt % ethyl acetate and 20.2 wt % water. Stream 2 may be recycled back into the mixer unit operation where the corn oil is extracted from DWG by subjecting the DWG to an ethyl acetate solvent to produce an extraction solution containing corn oil.

The same process consideration also applies for deoiling the TS that is available in the beer still bottoms.

### Example 10

### Generation of Power and Steam by Combustion of Deoiled DWG

Deoiled DWG has several advantages, one of which is the generation of power and steam by combustion.

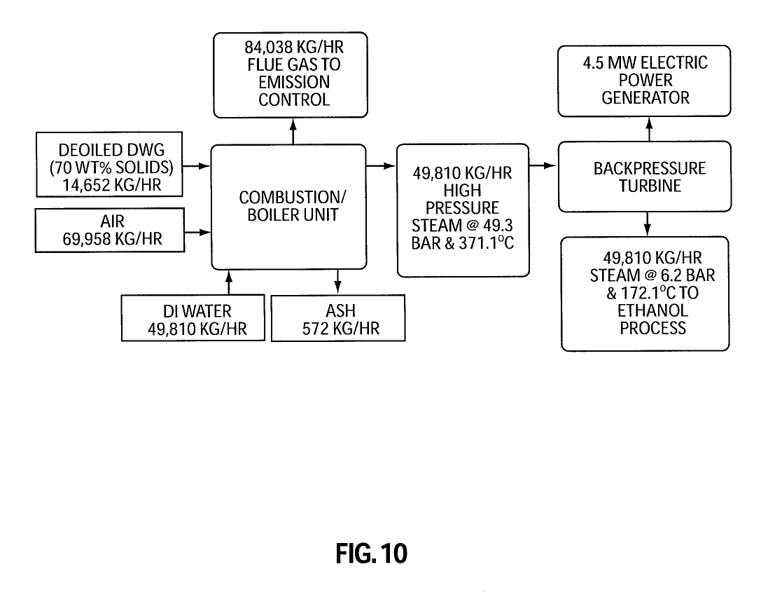

A stream of 14,652 kg/hr (32,303 lb/hr) of deoiled DWG with a moisture level of 30 wt % or 70 wt % solids is fed into a combustion/boiler unit such as a type offered by KMW Systems, Inc. (identified above) including but not limited to an English boiler available from KMW Systems. The unit is air blown and works at atmospheric pressure. A deionized water stream of 49,810 kg/hr and an air stream of 69,958 kg/hr are also fed into the combustion/boiler unit. The flue gas is directed through a compact high efficiency boiler where high pressure steam at 49.3 bar (715 psia) and 371.1° C. (700° F.) is produced. 84,038 kg/hr flue gas also exists the combustion/boiler unit toward an emission control unit. Ash is also produced at approximately 572 kg/hr. This steam is then fed into a backpressure turbine, which is coupled to an electric power generator. Typical steam turbines are available for this application from Dresser-Rand Murray (Houston, Tex.) and electric generators are readily available from international companies like General Electric (Fairfield, Conn.). As a result of the foregoing, 4.5 MWH electric power, commensurate with the pressure drop across the backpressure turbine, is produced. 49,810 kg/hr (109,812 lb/hr) of exhaust steam at 6.2 bar (90 psia) and 172.1° C. (342° F.) is also available and may be used for example, for the low pressure steam requirements of and is thus routed to the ethanol production process. The pressure can be specifically calibrated to maximize the use of this steam for the beer stills. A block diagram schematic of this application is shown in FIG. 10. In one embodiment, in order to produce higher electrical power, the steam pressure and temperature can be increased to 63.1 bar (915 psia) and 386.0° C. (727° F.) so as to produce 5.0 MWH. More preferably, the combustion/boiler unit, the steam pressure and temperature can be increased to 87.2 bar (1,265 psia) and 411.4° C. (773° F.), to increase output, including but not limited to an electrical power output increase from 5.0 to 5.6 MW.

### Example 11

### Gasification of Deoiled DWG

As indicated herein, deoiled DWG may also be gasified to produce syngas and other products therefrom.

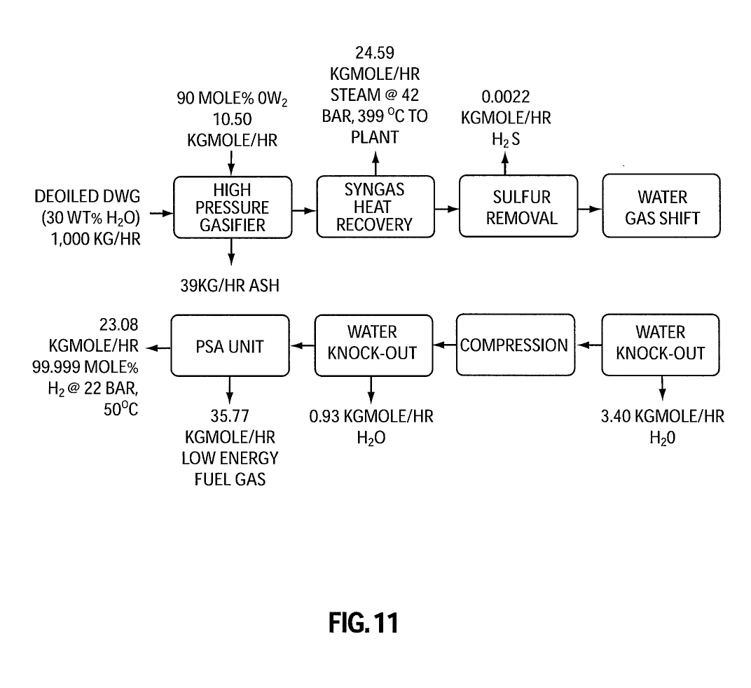

A block diagram schematic of the gasification process is shown in FIG. 11. 1,000 kg/hr of deoiled DWG with a moisture level of 30 wt % is fed along with an oxidant containing 10.50 kgmole/hr of 90 mole % oxygen and 10 mole % nitrogen into a gasifier. The gasifier is maintained at 4 bar and 962.67° C. 63.18 kgmole/hr of syngas is produced. 39 kg/hr of ash is also produced. Syngas composition according to the foregoing is detailed in TABLE 19.

TABLE 19
SYNGAS COMPOSITION

| Composition | Mole % |
|---|---|
| CH₄ | 0.01 |
| CO₂ | 17.14 |
| N₂ | 1.91 |
| CO | 23.67 |
| H₂ | 28.54 |
| H₂S (ppm) | 35 |
| H₂O | 28.72 |
| TOTAL | 100.00 |
| Pressure, Bar | 4.00 |
| Temperature, ° C. | 926.67 |
| HHV, BTU/SCF | 169 (wet), 237 (dry) |
| Kcal/M³ | 1,504 (wet), 2,109 (dry) |
| Flow, kgmole/hr | 63.18 |

The syngas is cooled to 420° C. in a boiler to raise 24.59 kgmole/hr of steam at 42 bar and 399° C. This steam may be returned to the ethanol production plant to supply power or other needs. Sulfur compounds are then removed from the syngas which typically contains 0.0022 kgmole/hr H₂S. This is done by the use of an iron based chelating agent that converts the sulfur compounds into iron pyrite. Manufacturers of this type of sulfur chelating agent include Merichem Sulfur-Rite (Houston, Tex.). The syngas is further cooled to 200° C. for conducting a water gas shift reaction. The moisture content in the shifted syngas is then knocked out at 40° C. by using direct or indirect condensers to produce approximately 3.40 kgmole/hr water. The shifted cool syngas has the composition noted below in TABLE 20.

TABLE 20
SHIFTED SYNGAS AFTER WATER KNOCK-OUT AT 40° C.

| Composition | Mole % |
|---|---|
| CH₄ | 0.02 |
| CO₂ | 40.84 |
| N₂ | 2.02 |
| CO | 2.29 |
| H₂ | 52.89 |
| H₂O | 1.94 |
| TOTAL | 100.00 |
| Pressure, Bar | 4.00 |
| Temperature, ° C. | 40.00 |
| HHV, BTU/SCF | 179 |
| Kcal/M³ | 1,593 |
| Flow, kgmole/hr | 59.78 |

The shifted syngas may then be compressed to 23 bar. The moisture content in the compressed syngas is again knocked out at 40° C. by use of direct and/or indirect condensers to produce approximately 0.93 kgmole/hr water.

23.08 kgmole/hr of H₂, which amounts for 73 mole % of the H₂ content in the shifted dry syngas can be recovered at 22 bar and 50° C. having a purity level of 99.999 mole % by a PSA unit along with a low energy fuel gas, at a rate of 35.77 kgmole/hr.

TABLE 21
H2 CONTENT IN SYNGAS

Name of Stream

| | Pure H₂ Produced | Low Energy Fuel Gas |
|---|---|---|
| Comp | Mole % | Mole % |
| CH₄ | — | 0.80 |
| CO₂ | — | 68.23 |
| N₂ | 0.0005 | 3.36 |
| CO | — | 3.54 |
| H₂ | 99.9995 | 23.42 |
| H₂O | — | 0.65 |
| TOTAL | 100.0000 | 100.00 |
| Pressure, Bar | 22.00 | 2.00 |
| Temperature, ° C. | 50.00 | 50.00 |
| HHV, BTU/SCF | 325 | 96 |
| Kcal/M³ | 2,892 | 854 |
| Flow, kgmole/hr | 23.08 | 35.77 |

### Example 12

### Corn Oil Extraction Comparison of Milled Corn, TS, DWG and DDGS

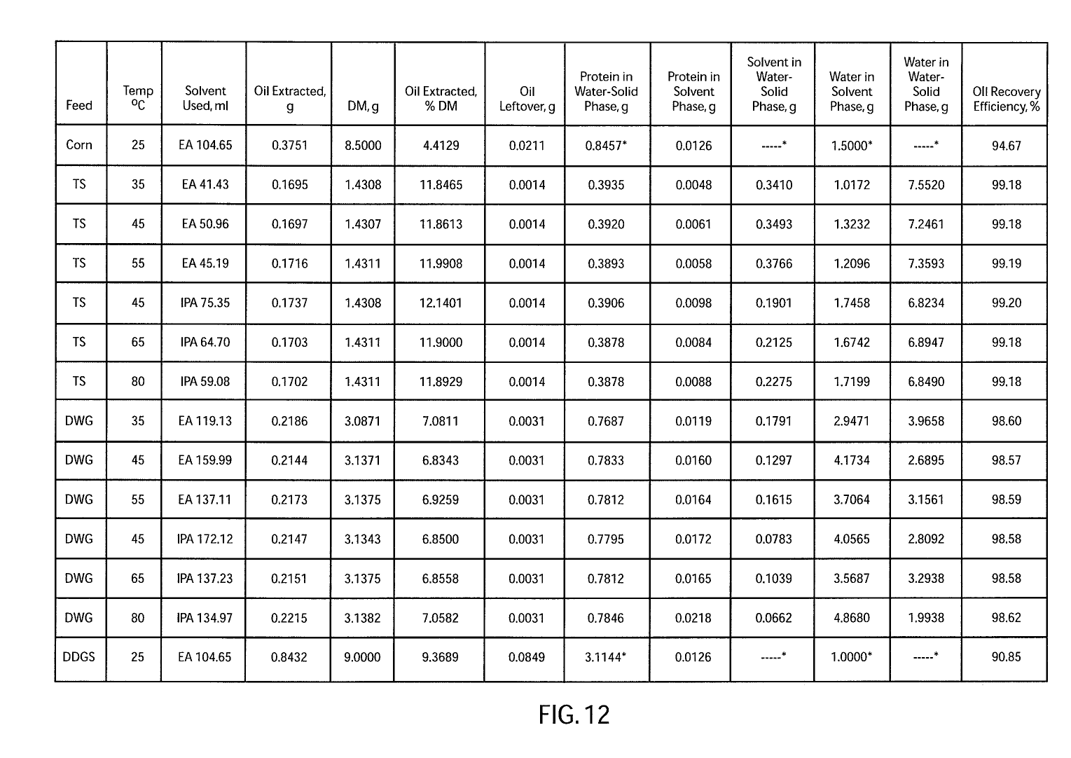

A comparison was performed for the extraction of corn oil from milled corn, TS, DWG and DDGS. The comparison includes analysis of each corn product/byproduct using oil extraction techniques described herein using ethyl acetate or isopropyl acetate at varying temperatures ranging from 25° C. to 80° C. The results are illustrated in FIG. 12.

As can be seen, using the various techniques described herein, oil can be recovered from milled corn, TS, DWG and DDGS with an efficiency ranging from greater than 90 wt % to greater than 99 wt %, with the greatest recovery percentage available from TS and DWG. These recovery rates are irrespective of the specific alkyl acetate solvent used and temperature applied.

Although various representative embodiments of this invention have been described above with a certain degree of particularity, those skilled in the art could make numerous alterations to the disclosed embodiments without departing from the spirit or scope of the inventive subject matter set forth in the specification and claims. Joinder references (e.g., attached, coupled, connected) are to be construed broadly and may include intermediate members between a connection of elements and relative movement between elements. As such, joinder references do not necessarily infer that two elements are directly connected and in fixed relation to each other. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to preferred embodiments, persons skilled in the art will recognize that changes may be made in form and detail without departing from the spirit and scope of the invention.

What is claimed is:

1. A process for extracting corn oil from corn in an ethanol production process, the process comprising obtaining a corn-based product from the ethanol production process, application of an alkyl acetate solvent to the corn-based product to extract oil so as to produce an extraction solution of at least corn-based product solids, oil, solvent and water, separating the extraction solution into a first phase containing solvent and oil and a second phase containing at least one of water and solids, separating the first phase from the second phase and removing the solvent from the oil.

2. The process for extraction corn oil of claim 1, wherein application of the alkyl acetate solvent occurs prior to fermentation in the ethanol production process.

3. The process of extraction of corn oil of claim 1, wherein the application of the alkyl acetate solvent occurs post fermentation in the ethanol production process and is applied to at least one byproduct of the fermentation process.

4. The process of claim 3, wherein the alkyl acetate is an azeotrope.

5. The process of claim 1, wherein the removed alkyl acetate solvent is recycled into the process.

6. The process of claim 1, wherein the deoiled second phase is concentrated by removal of a percentage of water.

7. The process of claim 6, wherein the deoiled second phase is concentrated to produce a filter cake.

8. The process of claim 7, wherein an ethanol azeotrope is applied to the filter cake to dissolve the remaining water and drying deoiled remaining solids by the application of carbon dioxide.

9. The process of claim 8, wherein ethanol carried by the carbon dioxide from the drying step is removed from the carbon dioxide via a condenser and the carbon dioxide is recycled back to the production process.

10. The process of claim 1, wherein the corn-based product comprises milled corn.

11. The process of claim 1, wherein the corn-based product comprises thick stillage.

12. The process of claim 1, wherein the corn-based product comprises distillers wet grain.

13. The process of claim 12, wherein the deoiled corn-based product comprises deoiled distillers wet grains and is concentrated by removal of a percentage of water and wherein syngas is produced from the concentrated deoiled distillers wet grain using a pressurized gasifier.

14. The process of claim 1, wherein the byproduct comprises distillers dried grains with solubles.

15. A process for production of co-products from an ethanol production facility comprising placing corn in a hammermill to produce a milled corn, transferring the milled corn into an extractor and blending the milled corn with an alkyl acetate solvent mixture to form a combined mixture for extracting an oil, separating the oil from the deoiled milled corn, removing the solvent mixture from the oil and deoiled milled corn and recycling the solvent mixture back into the extractor and transferring the deoiled milled corn to a fermentor to produce ethanol.

16. A process for production of co-products from an ethanol production facility comprising obtaining thick stillage from an ethanol fermentation unit, placing the thick stillage in a mixing unit with an alkyl acetate solvent mixture and blending to form a combined mixture for extracting an oil, allowing the combined mixture to settle into a first phase containing the solvent mixture and oil and a second phase containing deoiled thick stillage and water, separating the first phase and distilling the first phase to separate the oil from the solvent mixture and recycle the solvent mixture to the mixing unit, placing the second phase in a reboiler desorption unit to remove unseparated solvent for recycling, filtering the second phase containing deoiled thick stillage to produce deoiled distillers wet grains and deoiled thin stillage, concentrating the deoiled thin stillage to produce a retentate syrup and drying the combined syrup and deoiled distillers wet grains to produce oil free distillers dry grains with solubles.

17. A process for production of co-products from an ethanol production facility comprising obtaining distillers wet grain from a post fermentation process flow system, blending the distillers wet grain with an alkyl acetate solvent mixture to form a combined mixture for extracting an oil, allowing the combined mixture to settle into a first phase containing the solvent mixture and oil and a second phase containing deoiled distillers wet grain and water, separating the first phase and distilling the first phase to separate oil from the solvent mixture and recycle the solvent mixture, placing the second phase into a reboiler desorption unit to remove unseparated solvent for recycling, removing additional water content from the second phase and placing the deoiled, dewatered second phase into a combustion/boiler unit for the production of energy.

18. A process for production of co-products from an ethanol production facility comprising obtaining distillers dry grains with solubles from a post fermentation process flow system, blending the distillers dry grains with solubles with an alkyl acetate solvent mixture to form a combined mixture for extracting an oil, separating the oil from the solvent mixture and recycling the solvent mixture, removing the solvent remaining in the deoiled dry grains with solubles to produce oil free distillers dry grains with solubles and recycling the solvent remaining.

19. A process for drying a wet solid co-product containing an amount of moisture from an ethanol production process comprising obtaining an ethanol stream from the production process, converting the ethanol into an azeotrope, applying the ethanol azeotrope to the wet solid co-product to remove moisture and form an ethanol rich solid co-product, applying a stream of hot carbon dioxide gas from the ethanol production process to the ethanol rich solid co-product so as to remove ethanol from the solid co-product and form an ethanol-carbon dioxide stream, condensing the ethanol from the ethanol/carbon dioxide stream and recycling the carbon dioxide back into the production process, thereby leaving a dried solid co-product.

20. A system for removing oil from corn products prior to fermentation in an ethanol production facility comprising a hammermill for reducing whole corn to a milled corn and producing an output stream of milled corn; an extractor containing a solvent and in communication with the output stream from the hammermill capable blending of the milled corn with the solvent to form a solvent mixture and capable of producing a solvent mixture stream; and a solvent stripper in operable communication with an output solvent mixture stream from the extractor, the solvent stripper being capable of removing solvent from the solvent mixture to form a deoiled milled corn stream, the solvent stripper further being in operable communication with the extractor to provide at least a portion of the solvent stream to the extractor and in operable communication with a fermentor to provide a deoiled milled corn stream to the fermentor for the production of ethanol.

21. A system for removing oil from a corn product post fermentation in an ethanol production facility comprising an extractor containing a solvent and in operable communication with a fermentor, wherein the extractor is capable of receipt of an output thick stillage stream from the fermentor and forming a solvent mixture; a phase settler in operable communication with the extractor for receipt of the solvent mixture, the phase settler enabling the separation of a deoiled corn product phase from an oil in solvent phase; a distillation assembly in operable communication with the phase settler for receipt of an oil in solvent phase stream, the distillation assembly capable of separating the oil from the solvent, wherein the distillation assembly is in operable communication with the extractor to provide at least a portion of the solvent to the extractor; and a reboiler desorption unit operably attached to the phase settler for receipt of a deoiled corn product phase stream, the reboiler desorption unit being capable of removal of solvent remaining in the deoiled corn product phase, the reboiler desorption unit in operable communication with the extractor to provide at least a portion of the solvent stream to the extractor.

22. The system of claim 21, further comprising a filtration device operably attached to the reboiler desorption unit for receipt of the deoiled corn product stream, the filtration device capable of separating the deoiled thick stillage into an oil free distillers wet grain and an oil free thin stillage and in communication with a membrane for water removal and a drying device for producing an oil free distillers dried grain with solubles.

23. A system for removing oil from a corn product post fermentation in an ethanol production facility comprising an extractor containing a solvent and in operable communication with a fermentor, wherein the extractor is capable of receipt of an output distillers wet grain stream from centrifuging of a thick stillage from the fermentor and forming a solvent mixture; a phase settler in operable communication with the extractor for receipt of the solvent mixture, the phase settler enabling the separation of a deoiled corn product phase from an oil in solvent phase; a distillation assembly in operable communication with the phase settler for receipt of an oil in solvent phase stream, the distillation assembly capable of separating the oil from the solvent, wherein the distillation assembly is in operable communication with the extractor to provide at least a portion of the solvent to the extractor; and a reboiler desorption unit operably attached to the phase settler for receipt of a deoiled corn product phase stream, the reboiler desorption unit being capable of removal of solvent remaining in the deoiled corn product phase, the reboiler desorption unit in operable communication with the extractor to provide at least a portion of the solvent stream to the extractor.

24. The system of claim 23, further comprising a water removal device in operable communication with the reboiler desorption unit for receipt of the deoiled corn product stream, the water removal device being capable of removal of a percentage of water from the deoiled corn product stream.

25. The system of claim 24, further comprising a combustion device in operable communication with the water removal device to receive a deoiled distillers wet grain corn product stream, the combustion device being capable of generating energy output from the deoiled corn product stream.

26. The system of claim 24, wherein an optimum moisture level in the deoiled corn product stream for combustion is obtained and which comprises approximately 30 wt % moisture.

27. A system for removing oil from a corn product post fermentation in an ethanol production facility comprising an extractor containing a solvent and in operable communication with a fermentor, wherein the extractor is capable of receipt of an output distillers dried grains with solubles stream from the ethanol production facility and forming a solvent mixture; a phase settler in operable communication with the extractor for receipt of the solvent mixture, the phase settler enabling the separation of a deoiled corn product phase from an oil in solvent phase; a distillation assembly in operable communication with the phase settler for receipt of an oil in solvent phase stream, the distillation assembly capable of separating the oil from the solvent, wherein the distillation assembly is in operable communication with the extractor to provide at least a portion of the solvent to the extractor; and a reboiler desorption unit operably attached to the phase settler for receipt of a deoiled corn product phase stream, the reboiler desorption unit being capable of removal of solvent remaining in the deoiled corn product phase, the reboiler desorption unit in operable communication with the extractor to provide at least a portion of the solvent stream to the extractor.
